(19) United States
(12) Patent Application Publication
(10) Pub. No.: US 2011/0282075 A1
(43) Pub. Date: Nov. 17, 2011

(54) METHOD FOR PREPARING VITTATALACTONE

(75) Inventor: Bernhard BREIT, Gundelfingen (DE)

(73) Assignee: Albert-Ludwigs-Universitat Freiburg, Freiburg (DE)

(21) Appl. No.: 13/069,764

(22) Filed: Mar. 23, 2011

Related U.S. Application Data
(60) Provisional application No. 61/334,739, filed on May 14, 2010.

(51) Int. Cl. C07D 305/12

(52) U.S. Cl. 549/328

(57) ABSTRACT

The present invention relates to the chemical synthesis of vittatalactone, the aggregation pheromone of the striped cucumber beetle, Acalymma vittatum.

The present invention relates to the chemical synthesis of vittatalactone, the aggregation pheromone of the striped cucumber beetle, Acalymma vittatum.

### BACKGROUND OF THE INVENTION

Vegetable crops are an important commodity in the United States. The Northeastern states, in particular, have a high proportion of their vegetable crop industry invested in cucurbit crops, including squash, melons, cucumbers, and pumpkins.¹ The main insect pest of these cucurbit crops is the striped cucumber beetle, Acalymma vittatum, which also serves as the vector for Erwinia tracheiphila, a bacterium that causes a lethal wilt disease in cucurbits.² On this background, the demand for sustainable plant protection is high, since classical use of insecticides can be extremely expensive and additionally impacts pollinators and natural enemies.

It has been found by Smyth and Hoffmann that feeding by “male pioneers” results in a pheromone signal produced by the males, which attracts conspecifics.³ This aggregation behavior or concentrated feeding is a key component for the development of a bacterial wilt epidemic.⁴ The composition of the pheromone mixture secreted by the feeding male beetles was first analyzed by Morris and Francke and they discovered the main component to be Vittatalactone (1), accompanied by a minor compound, 12-Norvittatalactone (2).⁵ With 5 stereogenic centers, both β-lactones represent the most complex structures among physiologically active insect volatiles.

Recently, the total synthesis of the enantiomer of Vittatalactone based on an iterative allylic substitution concept was reported.⁶ By means of the enantiomer synthesis, it became possible to elucidate the absolute configuration of the natural product. Described herein is the evolution of the total synthesis, the determination of the absolute configuration of Vittatalactone through total synthesis of two eventual diastereomers, and finally a new and more convergent as well as scalable synthesis of the natural enantiomer of Vittatalactone (1).

### SUMMARY OF THE INVENTION

In a first aspect, the present invention relates to a method for preparing a compound of Formula (1)

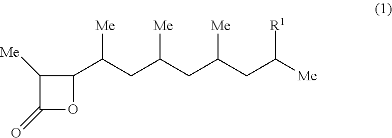

comprising the following steps:
a) reacting a compound of Formula (2)

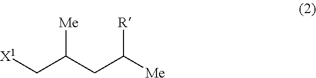

with a compound of Formula (3)

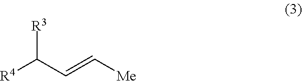

to obtain a compound of Formula (4)

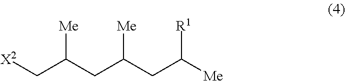

b) reacting the compound of Formula (4) with a compound of Formula (5)

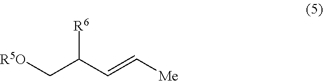

to obtain a compound of Formula (6)

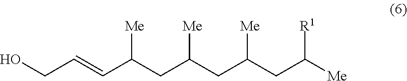

and
c) converting the compound of Formula (6) into the compound of Formula (1);
wherein
R¹ is hydrogen or methyl,
X¹ is halogen or —SR²,
R² is a hydrocarbon group,
X² is halogen or —SR²,
R³ is a leaving group,
R⁴ is a hydrocarbon group,
R⁵ is a protecting group, and
R⁶ is a leaving group.

In a second aspect, the present invention relates to a method for preparing a compound of Formula (1)

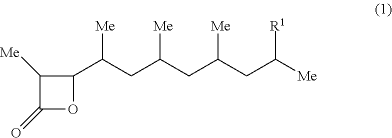

wherein R¹ is hydrogen or methyl, comprising the following steps:
a) converting a compound of Formula (18)

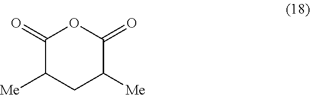

into a compound of Formula (4)

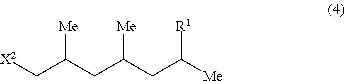

b) reacting the compound of Formula (4) with a compound of Formula (5)

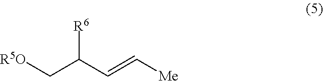

to obtain a compound of Formula (6)

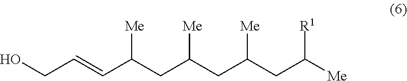

and
c) converting the compound of Formula (6) into the compound of Formula (1);
wherein
X² is halogen or —SR²,
R² is hydrocarbon group;
R⁵ is a protecting group, and
R⁶ is a leaving group.

In yet another aspect, the present invention relates to the use of a compound of Formula (18)

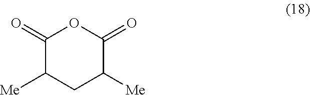

for the preparation of vittatalactone or norvittatalactone.

### BRIEF DESCRIPTION OF THE FIGURES

FIG. 1 shows a comparison of NMR spectra of natural Vittatalactone (upper spectrum),⁵ β-lactone (−)-1b (spectrum in the middle) and β-lactone 1a (lower spectrum, dr 92:8).

### DETAILED DESCRIPTION OF THE INVENTION

### First Generation Synthesis

In a first aspect, the present invention relates to a method for preparing a compound of Formula (1)

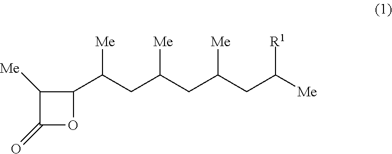

wherein R¹ is hydrogen or methyl. Preferably R¹ is methyl (vittatalactone). If R¹ is hydrogen, the compound is norvittatalactone. The compound of Formula (1) may be present in different configurations, which is illustrated on the basis of vittatalactone. The compound of Formula (1a) is the naturally occurring vittatalactone.

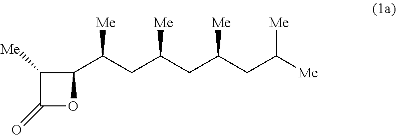

The compounds of Formula (1b) and (1c) represent stereoisomers of the naturally occurring vittatalactone.

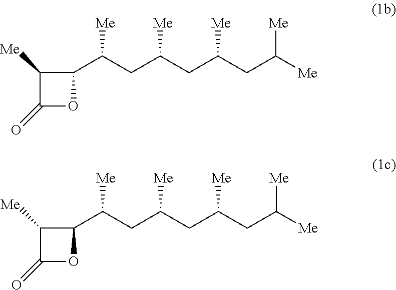

The method of the first aspect of the invention can be used to prepare any of the stereoisomers of the compound of Formula (1). It is preferred, however, that the method is used for the preparation of vittatalactone or norvittatalactone having the stereochemical configuration as shown in Formula (1a) for vittatalactone.

The terms “Me” and “Et”, as used herein, denote methyl and ethyl, respectively. The term “alkyl” preferably refers to C₁₋₂₀ alkyl.

According to the method of the first aspect of the invention, step a) of the method comprises reacting a compound of Formula (2)

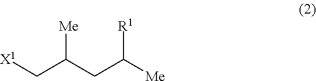

with a compound of Formula (3)

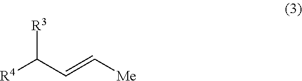

to obtain a compound of Formula (4)

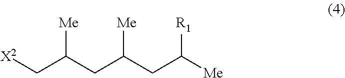

X¹ is halogen or —SR², with R² being a hydrocarbon group. The halogen may be selected from the group consisting of Cl, Br and I. Preferably, X′ is Br or I; most preferably X¹ is Br.

R² is preferably an aliphatic hydrocarbon group, e.g. substituted or unsubstituted, straight chain or branched, alkyl, alkenyl or alkinyl, or cycloalkyl. More preferably, R² is a lower alkyl chain, which may be straight chain or branched, and which may be substituted or unsubstituted. Most preferably, R² is a lower alkyl chain, in particular methyl, ethyl, n-propyl, isopropyl, n-butyl, isobutyl, n-pentyl or n-hexyl.

As used herein the term “lower alkyl” refers to an alkyl group having one to six carbon atoms. The term “lower alkenyl” refers to an alkenyl group having two to six carbon atoms.

R³ may be any suitable leaving group. As used herein, the term “leaving group” denotes an atom or group that becomes detached from the rest of a molecule during a reaction. Suitable leaving groups are generally known to one of ordinary skill. Known leaving groups include esters of carboxylic acids, of sulfonic acids and of phosphoric acid; halogenides, and carbonates. Examples are nonaflates (—OSO₂C₄F₉), triflates (—OSO₂CF₃), fluorosulfonates (—OSO₂F), tosylates, mesylates, iodides, bromides and the like. Preferred leaving groups according to this invention include ortho-diphenylphosphanyl benzoic acid ester (o-DPPB), picolinate, pentafluorobenzoate, mesylate and the like.

R⁴ may be any aliphatic or aromatic hydrocarbon group. Aliphatic groups include alkyl, alkenyl, alkinyl, cycloalkyl, heterocycloalkyl, which may branched or unbranched (if applicable), and which may be substituted or unsubstituted. Preferred aliphatic groups are lower alkyl, e.g. methyl, ethyl, n-propyl, isopropyl, n-butyl, isobutyl, n-pentyl or n-hexyl. Most preferably, R⁴ is ethyl. Alternatively, R⁴ may be aryl, such as phenyl, naphthyl, or heteroaryl.

X² may be the same as X¹ or be different. X² is halogen or —SR², with R² being a hydrocarbon group. The possible meanings and preferred embodiments of X² are the same as described above for X¹ herein.

The compound of Formula (2) can be prepared via various routes. The compound of Formula (2) may be prepared from a compound of Formula (7)

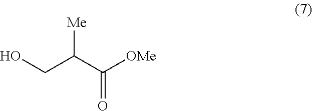

by converting it into a monoprotected diole, which is available from the compound of Formula (7) through alcohol protection and hydride reduction of the ester functionality. The monoprotected diole may then be activated as the tosylate and submitted to the conditions of a copper catalyzed reaction with isopropyl magnesium bromide to furnish PMB-ether in good yield (see Scheme 2 infra). The preparation of the compound of Formula (2) may then be performed as a one-pot procedure as depicted in Scheme 2.

Alternatively, the compound of Formula (2) may be prepared from a compound of Formula (8), from a compound of Formula (9), or from a compound of Formula (10), as described in Scheme 3 infra.

The compound of Formula (2) may be a compound of Formula (2a)

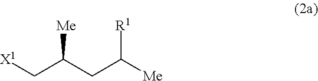

or a compound of Formula (2b)

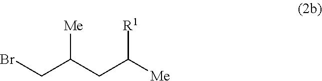

or a compound of Formula (2c)

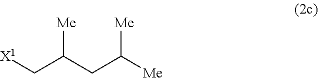

or a compound of Formula (2d)

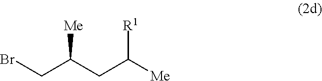

or a compound of Formula (2e)

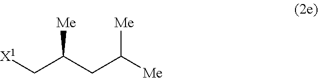

or a compound of Formula (2f)

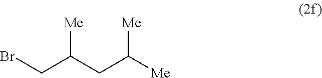

or a compound of Formula (2g)

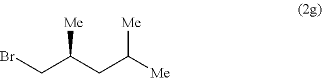

Most preferably, the compound of Formula (2) is the compound of Formula (2g)

Compound (3) is preferably a compound of Formula (3a)

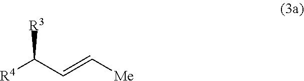

Step a) of the method of the first aspect of the invention is usually effected in several steps. Accordingly, step a) preferably comprises

(i) reacting a compound of Formula (2)

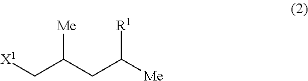

with the compound of Formula (3) to obtain a compound of Formula (11)

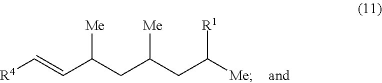

(ii) converting the compound of Formula (11) into the compound of Formula (4).

R⁴ has the same meaning as defined above.

In one embodiment, step a) comprises

(i) reacting a compound of Formula (2f)

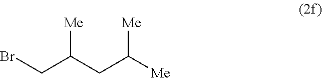

with the compound of Formula (3) to obtain a compound of Formula (11b)

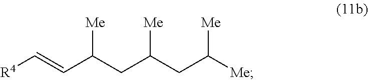

and
(ii) converting the compound of Formula (11b) into a compound of Formula (4f)

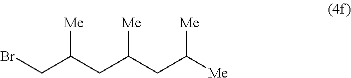

This type of direct Grignard synthesis is described in detail in Scheme 5 infra.

The compound of Formula (11) is preferably a compound of Formula (11a)

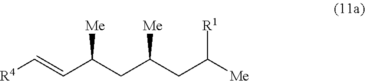

or a compound of Formula (11b)

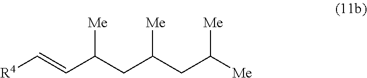

or a compound of Formula (11c)

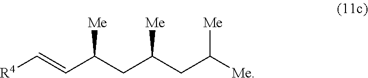

The compound of Formula (4) is preferably a compound of Formula (4a)

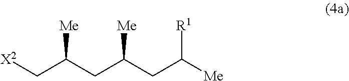

or a compound of Formula (4b)

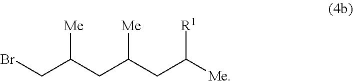

or a compound of Formula (4c)

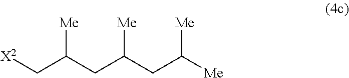

or a compound of Formula (4d)

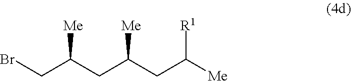

or a compound of Formula (4e)

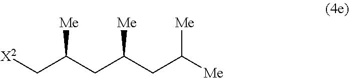

or a compound of Formula (4f)

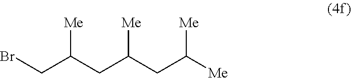

or a compound of Formula (4g)

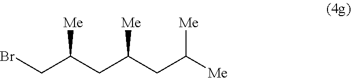

Most preferably, the compound of Formula (4) is the compound of Formula (4g)

The compound of Formula (5) is preferably provided by converting a compound of Formula (12)

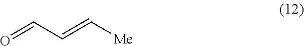

into a compound of Formula (13)

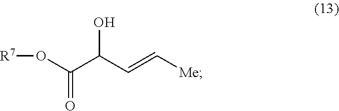

(ii) converting the compound of Formula (13) into a compound of Formula (14)

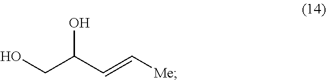

(iii) converting the compound of Formula (14) into a compound of Formula (15)

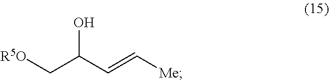

and
(iv) converting the compound of Formula (15) into the compound of Formula 5.

R⁵ is a protecting group, and R⁷ is an alkyl group. As used herein, the term “protecting group” denotes a chemical group which is introduced into a molecule by chemical modification of a functional group in order to obtain chemoselectivity in a subsequent chemical reaction. Preferably R⁵ is an alcohol protecting group. Such alcohol protecting groups are known to those of skill in the art. Suitable alcohol protecting groups are trityl, tetrahydropyranyl, or silyl ethers (such as trimethylsilyl, tert-butyldimethylsilyl, tert-butyldimethylsilyloxymethyl, triisopropylsilyl, or tert-butyldiphenylsilyl (TBDPS). Suitable alcohol protecting groups are further disclosed in “Greene's Protective Groups in Organic Synthesis”, 2007, John Wiley & Sons, 4th edition, ISBN-10: 0471697540; ISBN-13: 978-0471697541, the disclosure of which is incorporated herein in its entirety.

R⁷ is preferably a lower alkyl group, such as methyl, ethyl, n-propyl, isopropyl, n-butyl, isobutyl, n-pentyl or n-hexyl.

The reaction may be performed as depicted in detail in Scheme 6 infra.

Step b) of the method of the first aspect of the invention is preferably performed under the conditions described in Scheme 7 infra. That is, a tetrabutylammoniumfluoride coupled compound is generated in a first step, which is then converted into the compound of Formula (6) in a second step.

Finally, in step c) the compound of Formula (6) is reacted into a compound of Formula (1). This may be effected by

(i) converting the compound of Formula (6) into a compound of Formula (16)

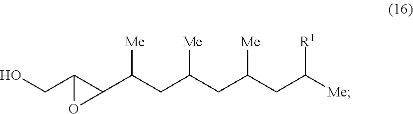

(ii) converting the compound of Formula (16) into a compound of Formula (17)

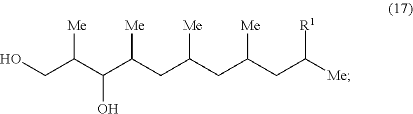

and
(iii) converting the compound of Formula (17) into the compound of Formula (1).

These final steps to obtain the compound of Formula (1) may be performed as described in detail in Scheme 8 infra.

Also the naturally occurring vittatalactone of Formula (1a) can be prepared by the method of the first aspect of the invention. An exemplary synthesis is shown in the following:

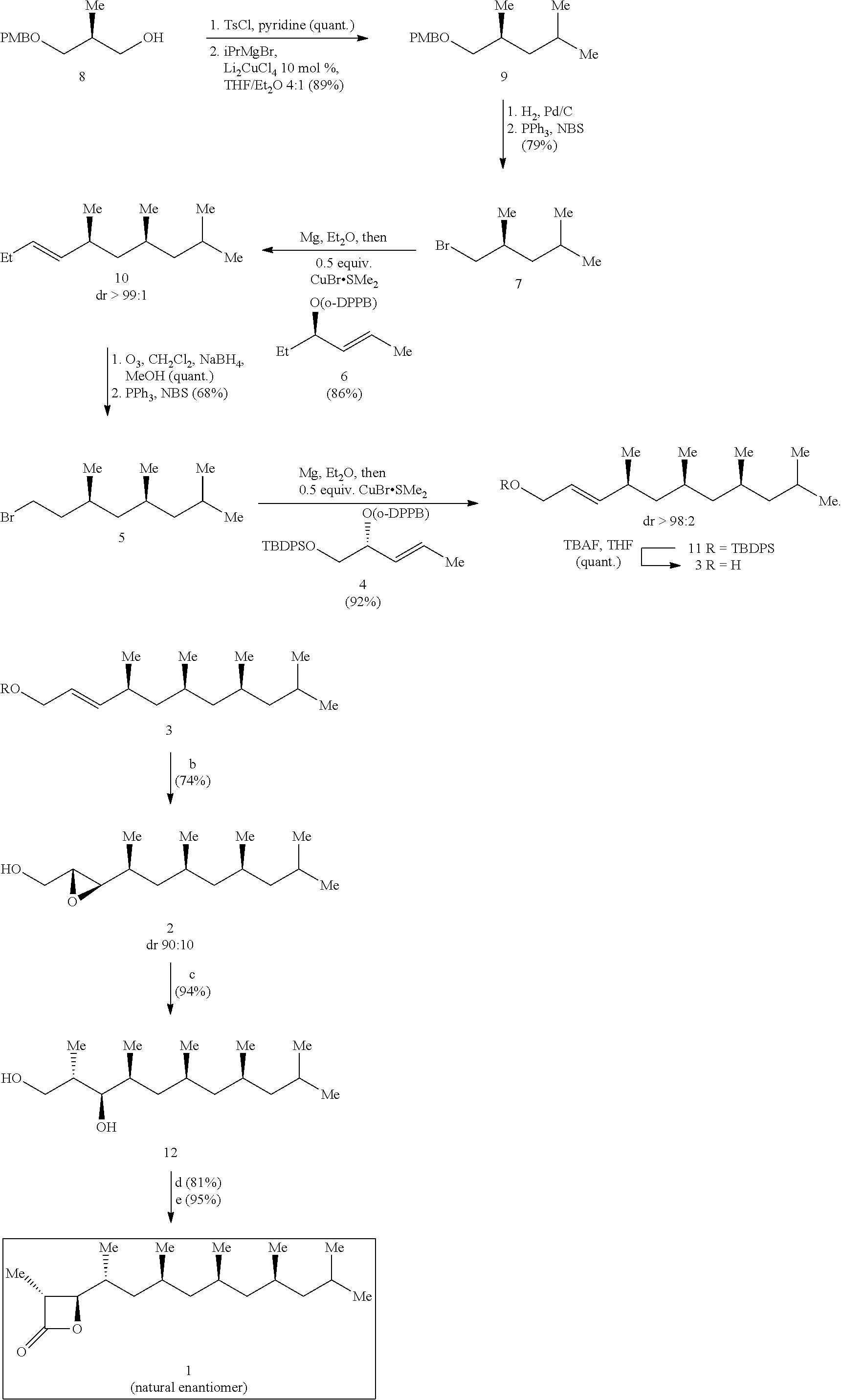

### Second Generation Synthesis

According to the second aspect of the invention, the compound of Formula (1) is prepared starting from a compound of Formula (18). This method is especially suitable for synthesizing naturally occurring Vittatalactone, which is a compound of Formula (1a)

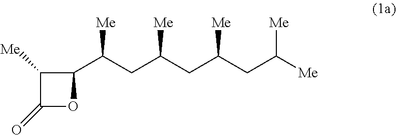

Preferably, the compound of Formula (18) is a compound of Formula (18a). Accordingly, in a preferred embodiment of the method of the second aspect of the invention step a) of the method comprises

converting the compound of Formula (18a)

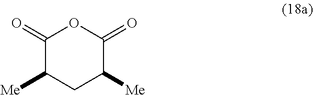

into the compound of Formula (4a)

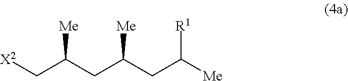

wherein X is halogen or —SR², and R² has the same meaning as defined supra. The bold presentation of the two bonds in formula (18a) means that both methyl groups have cis-configuration, which is preferred.

This reaction is preferably effected through several steps. That is, the method of the second aspect of the invention preferably comprises

(i) converting the compound of Formula (18a) into a compound of Formula (19a)

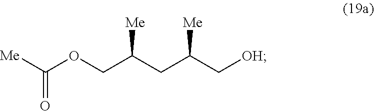

(ii) converting the compound of Formula (19a) into a compound of Formula (20a)

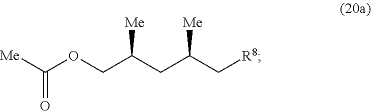

and
(iii) converting the compound of Formula (20a) into the compound of Formula (4a).

R⁸ is a leaving group, and the preferred embodiments of R³ and R⁶ as defined supra apply to R⁷ mutatis mutandis. Preferred details of the reaction can be performed as outlined in Scheme 10 infra.

Step b) of the method of the second aspect of the invention preferably comprises reacting the compound of Formula (4a) with a compound of Formula (5a)

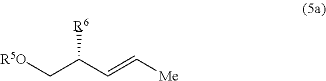

to obtain a compound of Formula (6a)

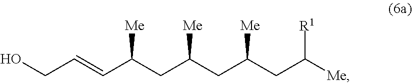

wherein R⁶ is a leaving group, and R⁵ is a protecting group. Preferred meanings of R⁵ and R⁶ are as defined above.

Finally, step c) of the method of the second aspect of the invention is preferably carried out by

(i) converting the compound of Formula (6a) into the compound of Formula (21a)

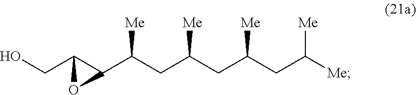

(ii) converting the compound of Formula (21a) into a compound of Formula (22a)

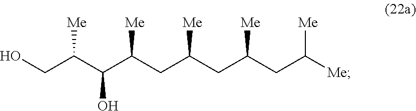

(iii) converting the compound of Formula (22a) into a compound of Formula (23a)

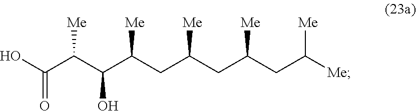

and
(iv) converting the compound of Formula (23a) into (+)-vittatalactone having the following structure:

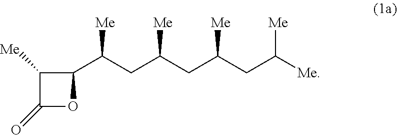

Preferred details of this reaction are described in Scheme 11 below. These reaction conditions are preferred embodiments of the present invention.

Finally, the use according to the third aspect of this invention corresponds to the preferred embodiments described above with respect to the method of the second embodiment of the invention. That is, the preferred embodiments of the above-described method of the invention are entirely applicable to the use of the third aspect of the invention.

The following examples further illustrate the invention.

### EXAMPLES

### Example 1

### First Generation Synthesis of Vittatalactone

### Chart 1. Structures of Vittatalactone (1), 12-Norvittatalactone (2) and Ebelactone A (3).

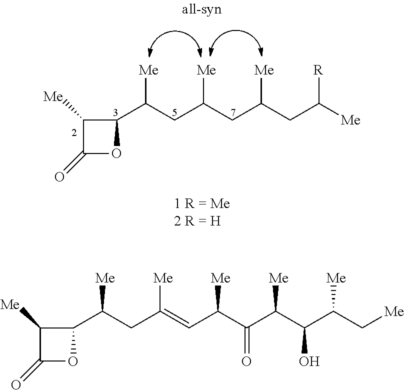

Target Structure Assignment. The structure published by the Francke group did not include the stereogenic information on the methyl branched side chain (Chart 1). By NMR analysis of the Mosher ester derivative of the corresponding hydroxy acid they assigned the stereocenters at C2 and C3 of (1) to be (2R,3R).⁵ Unknown at this point was the relative configuration of the alcohol at C3 and the methyl group on C4, while Morris and Francke referred to the close structural relationship of 1 to Ebelactone A (3), isolated from a strain of Streptomyces, which is an inhibitor of esterases, lipases, and N-formylmethionine aminopeptidases.⁷ This lactone exhibits an anti-relationship between C3 and C4 and results from polyketide biosynthesis, involving propionate-derived subunits, which also seems likely for Vittatalactone (1).⁸

In addition to the findings of Morris and Francke, we compared the NMR shifts of the methylene protons at C5 and C7 with the NMR data obtained for other deoxypropionate derived compounds and this examination resulted in the presumption of an all-syn configuration referring to the methyl groups.^{6,9} On this background, we chose the syn- and anti-lactones 1a and 1b (Scheme 1), respectively, as our target structures. This would enable us to derive both possible diastereomers of the natural product from an enantioselective Sharpless epoxidation of a common intermediate, allylic alcohol 5. Hence, such a divergent approach would then allow us to determine the absolute configuration of Vittatalactone.

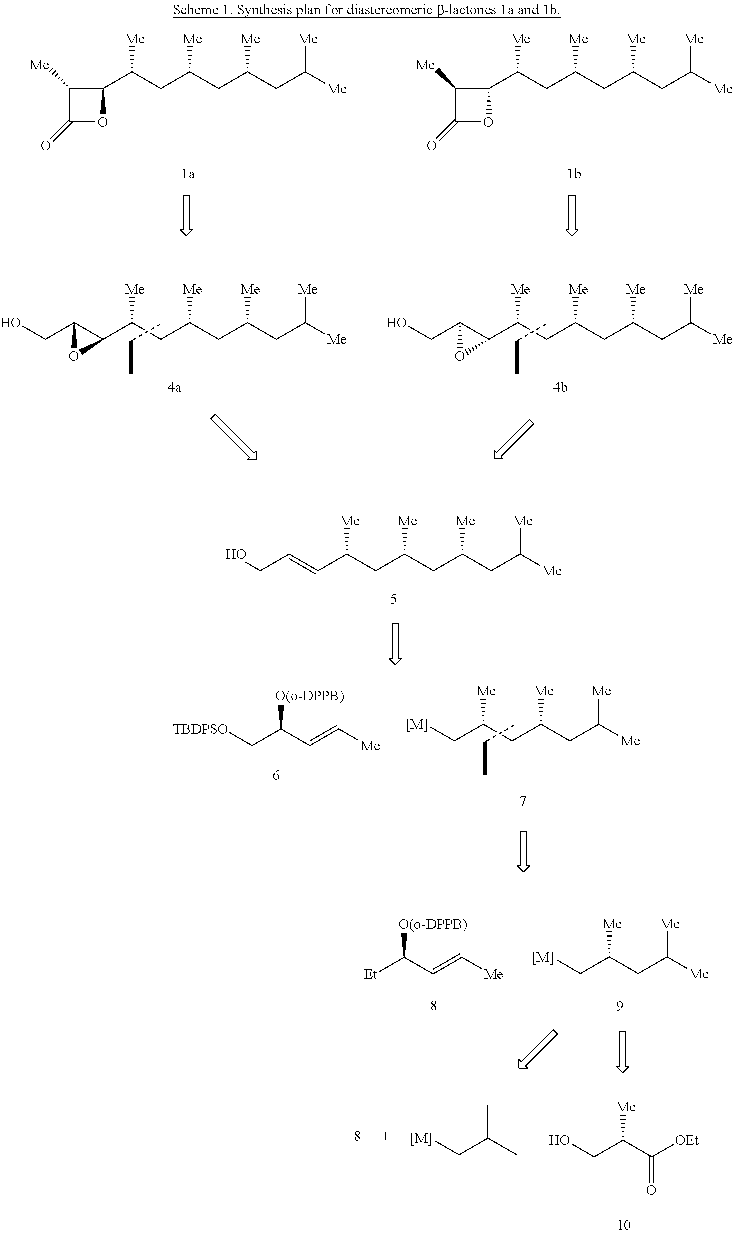

Results and Discussion

First Generation Retrosynthetic Approach

The lactones 1a and 1b would be accessible from the epoxides 4a and 4b, respectively, by regioselective ring opening with a methyl copper reagent (Scheme 1). We then targeted the allylic alcohol 5 as the common intermediate for the epoxide synthesis. This polyketide structure could be assembled via the iterative o-DPPB-directed allylic substitution method developed in our group.¹⁰ We have successfully demonstrated this principle in several syntheses,^{6,11} and Vittatalactone provided another challenging test case to explore the generality of this concept. Thus, the common intermediate 5 could stem from a directed allylic substitution of functionalized o-DPPB-ester 6 and dideoxypropionyl organometallic reagent 7. This step could set the third tertiary stereogenic center of the trideoxypropionate unit and would simultaneously introduce the allylic alcohol functionality.

The dideoxypropionyl organometallic reagent 7 in turn could be derived from a second directed allylic substitution employing the deoxypropionate building block 8 and the organometallic reagent 9. This first tertiary stereogenic center may again be generated on performing an allylic substitution on o-DPPB-ester 8 with an isobutyl Grignard reagent. Alternatively, it may stem from the commercially available Roche ester 10.¹²

Synthesis of (−)-Vittatalactone and Structure Elucidation

Preliminary Study on Allylic Substitution and Synthesis of Dideoxypropionyl Bromide (−)-19. Our synthesis started with monoprotected diol 11,¹³ which is available in two steps from the Roche ester 10 through alcohol protection and hydride reduction of the ester functionality. Alcohol 11 was activated as the tosylate 12 and submitted to the conditions of a copper catalyzed sp³-sp³-cross coupling reaction with isopropylmagnesium bromide (Scheme 2) to furnish PMB-ether 13 in good yield.^{14,15} The preparation of the suitable Grignard precursors, bromide 14 and iodide 15 was performed as a one-pot procedure due to the high volatility of the intermediate alcohol and the bromide and iodide, respectively.

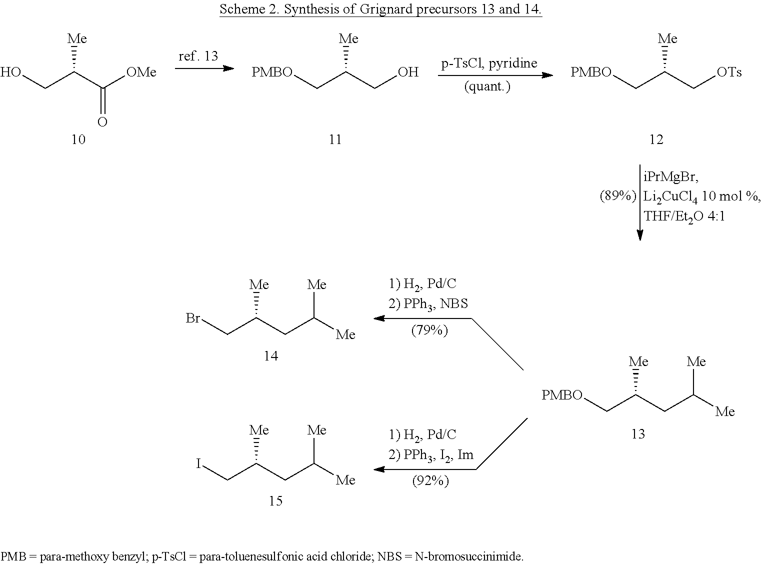

Additionally, alternative synthetic approaches towards bromide 14 were studied based on methodology developed in this group. Thus, subjection of allylic o-DPPB-ester 8 to the conditions of the copper-mediated allylic substitution with isobutyl magnesium bromide furnished the corresponding syn-S_{N}2′-product.^{10d} Subsequent ozonolysis followed by a reductive workup and bromination gave bromide 14 in overall good yields. Two alternative syntheses of 14 rely on the zinc-catalyzed enantiospecific sp³-sp³-coupling developed recently in our group.¹⁶ Thus, starting from D-lactic acid derived triflate 16 bromide 14 was accessible in three steps. Notably, the yield in the zinc-catalyzed coupling step with isobutyl magnesium chloride was essentially quantitative. Alternatively, one may start from triflate 17 which is readily available through diazotization of L-leucine. Coupling with methyl magnesium chloride, LiAlH₄ reduction and bromination provided a third efficient pathway towards bromide 14.

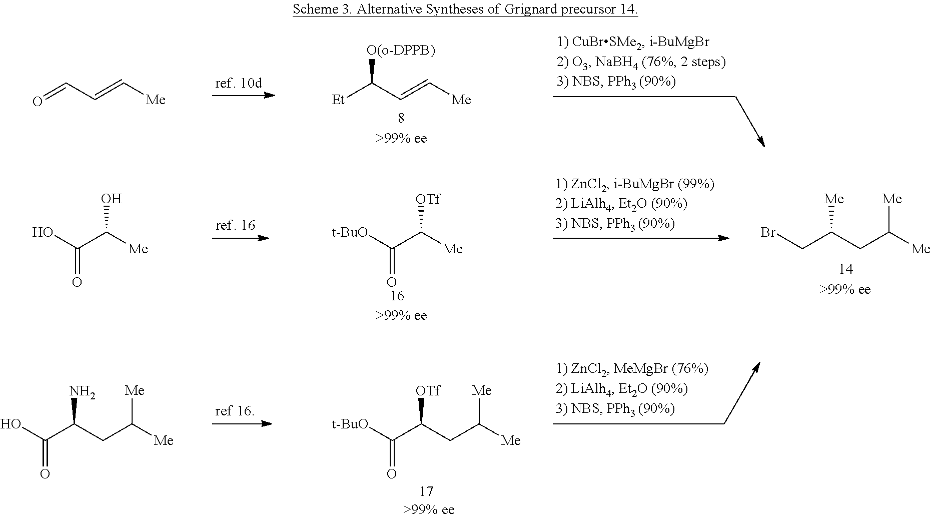

Directed allylic substitution using the o-DPPB-ester 8^{10c,d} as coupling partner was examined first with the iodide 15, according to the procedure used in the synthesis of 4,6,8,10,16,18-hexamethyldocosane (Scheme 4).^{11c,d} Thus, iodine-lithium exchange was effected upon treatment of iodide 15 in diethylether with two equivalents of tert-butyllithium at −100° C. The thus-obtained organolithium species was transmetallated to magnesium upon warming to room temperature with magnesium dibromide etherate. The resulting Grignard reagent was subjected to the conditions of the directed allylic substitution with o-DPPB-ester 8 to furnish the S_{N}2′ substitution product 16 in good yield and diastereoselectivity.

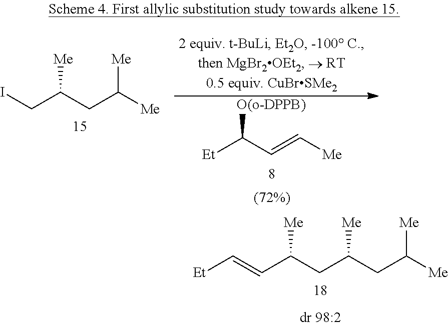

Although this procedure works quite well on a small (1-2 mmol) reaction scale, it has limitations on larger scale. Thus, the use of pyrophoric tert-butyllithium in large quantities is undesirable as is the requirement of the extreme low temperature conditions of −100° C. In order to allow for a more convenient and scalable allylic substitution process, we looked at the direct Grignard synthesis starting from bromide 14. Indeed, the bromide 14 was reduced with magnesium (turnings, etched three times with dibromoethane and thoroughly washed with diethylether) to the corresponding Grignard reagent which was directly used in the o-DPPB-directed allylic substitution with unfunctionalized o-DPPB-ester 8 to furnish dideoxypropionate 18 in excellent diastereoselectivity (>99:1) and yield (86%, Scheme 5). In order to incorporate the third propionate unit, we initiated the next iteration, which began with ozonolysis of alkene 18 and a reductive workup to furnish the corresponding alcohol. Mukaiyama redox condensation provided bromide (+)-19.¹⁷

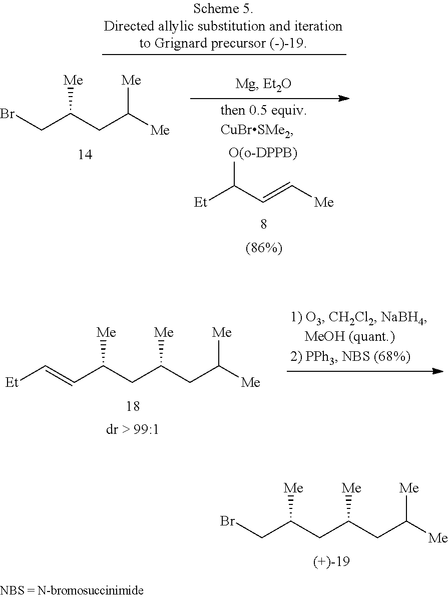

Enantioselective Preparation of o-DPPB-esters (R)- and (S)-6. For installation of the third methyl-branched tertiary stereogenic center with concomitant assembly of the allylic alcohol in 5 we required access towards oxygen-functionalized o-DPPB-esters 6.

Thus, crotonaldehyde was treated with HCN in the presence of an (R)-oxynitrilase readily obtained from grinding and scouring of bitter almonds,¹⁸ which gave the (R)-cyanohydrin with high levels of enantioselectivity (>96% ee).¹⁹ Subjecting to the conditions of a Pinner reaction furnished the ethyl ester 20 (Scheme 6).²⁰ The reduction with lithium aluminum hydride led to diol 21 and subsequent silylation furnished the silylether 22 on a multigram scale.²¹ Applying the standard Steglich esterification protocol²² with ortho-diphenylphosphanylbenzoic acid (o-DPPBA)²³ provided o-DPPB-ester (R)-(+)-6 quantitatively. Crystallization of this product improved the enantiopurity to greater than 99% ee. In order to obtain the requested (S)-enantiomer of 6 one could apply a corresponding (S)-oxynitrilase. However, such enzymes are far more difficult to access.²⁴ Hence, we looked at a Mitsunobu inversion protocol, which ideally would use o-DPPBA itself as the nucleophile.²⁵ Since o-DPPBA is both a carboxylic acid and a phosphine we expected this to be a non trivial reaction because the reagent triphenylphosphine as well as o-DPPBA may react with the azodicarboxylate electrophile. Interestingly, we observed a clean Mitsunobu reaction of the allylic alcohol 22 with o-DPPBA to furnish the corresponding (S)-(−)-enantiomer of o-DPPB-ester 6 in good yield (81%). After recrystallization (S)-(−)-6 was obtained in >99% enantiopurity.

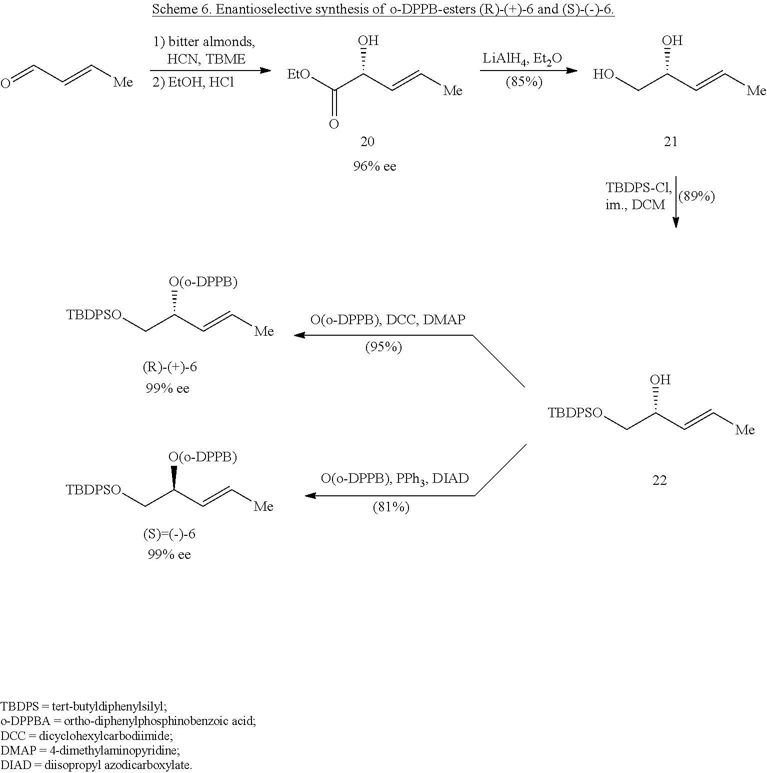

Final Steps Towards β-lactones 1a and 1b. With the bromide (+)-19 and o-DPPB-ester (S)-(−)-6 in hand, we could approach the construction of the central allylic alcohol intermediate 5 in our divergent synthesis strategy towards both eventual diastereomers of Vittatalactone. Thus, bromide (+)-19 was transferred into the corresponding Grignard reagent upon treatment with magnesium in diethylether,²⁶ and subjected to the conditions of the o-DPPB-directed allylic substitution with the oxygen-functionalized o-DPPB-ester (S)-(−)-6 furnished the allyl silyl ether 23 in excellent yield and diastereoselectivity (Scheme 7).²⁷ Noteworthy, only 1.05 equivalents of the valuable bromide (+)-19 were necessary to achieve full conversion in the directed allylic substitution, which renders this reaction a valuable tool in enantioselective carbon skeleton construction and even suitable for the use as a fragment coupling in the course of a convergent total synthesis.^{11b} Finally, fluoride mediated desilylation of 23 liberated the key allylic alcohol (−)-5 quantitatively.

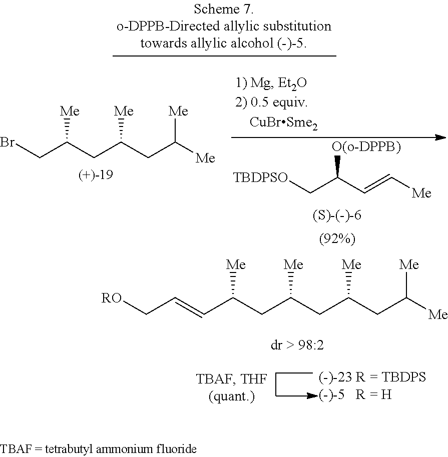

Transformation of allylic alcohol (−)-5 towards the two diastereomeric target structures 1a and 1b commenced with the catalyst-controlled stereoselective Sharpless epoxidation employing D- and L-diethyltartrate, respectively (Scheme 8).²⁸ Both diastereomeric epoxy alcohols 4a and (−)-4b were obtained in good yields and diastereoselectivity, even for the mismatched case towards (−)-4b.

The carbon skeleton of vittatalactone was completed by addition of cyanodimethylcuprate to give the 1,3-diols 24a and (+)-24b, respectively, in good yield.²⁹ At this stage, the minor diastereomer of (+)-24b (being 24a) could be separated by column chromatography to provide (+)-24b in a diastereomeric ratio of greater than 98:2. A highly selective oxidation of the primary alcohol of the 1,3-diols 24 towards the corresponding β-hydroxy aldehydes was accomplished applying 4-methoxy-TEMPO/hypochlorite in good yields,³⁰ even though the proximity of the secondary alcohol renders it a rather difficult synthetic operation.³¹ Pinnick oxidation then completed the oxidation step to furnish the respective β-hydroxy acids.³² The final ring closure was initiated with tosyl chloride in pyridine to give desired β-lactones 1a and (−)-1b in good to high yields.33

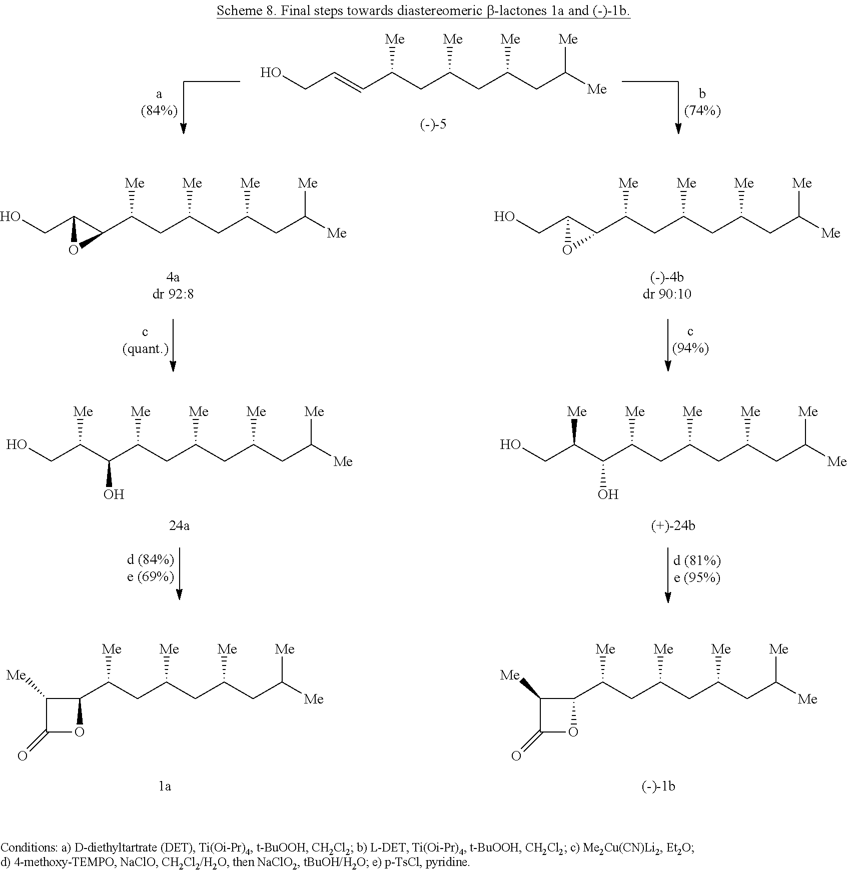

### Example 2

### Second Generation Synthesis

Second Generation Synthesis of Natural (+)-Vittatalactone

With the knowledge of the correct absolute configuration of Vittatalactone we decided to prepare the natural stereoisomer by way of a modified and more convergent synthetic strategy which would allow for the synthesis of enough material for biological studies. Thus, a commercially available starting material for the bromide 19 is the meso-anhydride 25 (Scheme 10). Enzymatic desymmetrization of the corresponding meso-diol has been described.³⁴

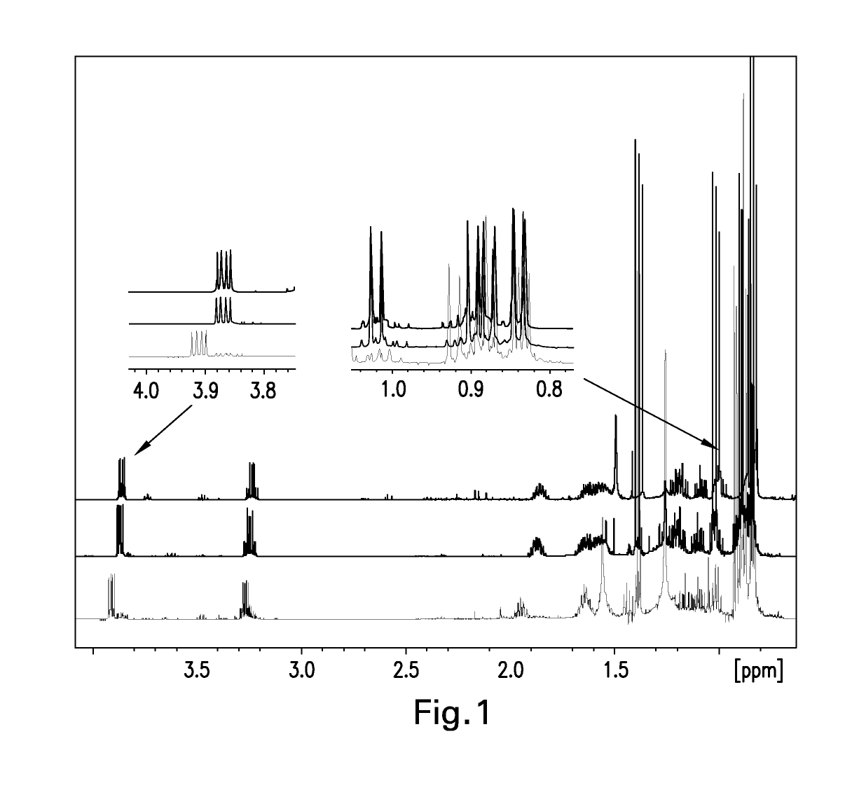

Structure Elucidation of Vittatalactone. Both diastereomeric lactones 1a and (−)-1b were subjected to NMR spectroscopic analysis. Comparison of the proton and carbon NMR data with those of the natural material isolated by the Francke group showed a complete match for β-lactone (−)-1b (FIG. 1).⁵ Hence, (−)-1b possesses the correct relative configuration of the natural product while, compared with the Mosher ester structure analysis of Francke et al., β-lactone (−)-1b is 2S,3S configured and should therefore be the enantiomer of natural vittatalactone.

Thus following the protocol developed by Mori et al. commercially available meso-anhydride 25 was reduced with lithium aluminium hydride to the corresponding meso-diol. Subsequent desymmetrization with lipase AK and vinylacetate furnished the monoacetate 26 in high enantiomeric purity (97% ee)³⁵ in 80% yield. The hydroxy group was activated as a tosylate 27, and subjected to the conditions of a copper-catalyzed sp³-sp³-cross-coupling reaction employing an excess of isopropyl magnesium bromide. The resulting alcohol 28 was transferred to the bromide (−)-19 via Mukaiyama redox condensation¹⁷ Thus, with this route starting from commercially available meso-anhydride 24 the desired dideoxypropionyl bromide (−)-19 was readily available in large scale in four steps and 62% overall yield employing only one chromatographical purification step.

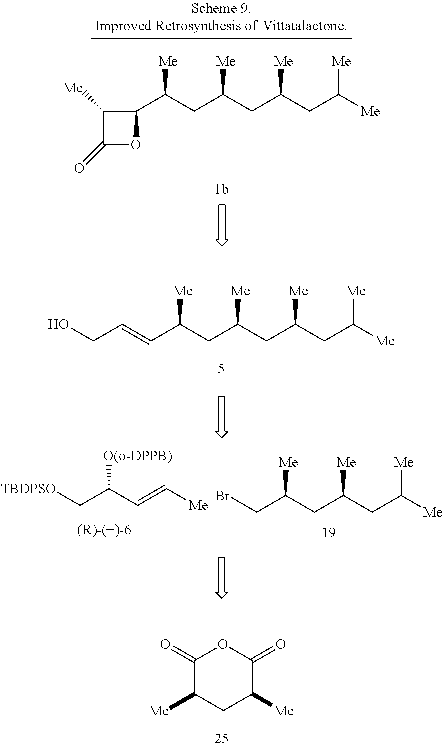

The final steps towards natural Vittatalactone were straightforward according to the enantiomer synthesis (Scheme 11). Allylic substitution with o-DPPB-ester (R)-(+)-6 and deprotection afforded the allylic alcohol (+)-5 with complete diastereoselectivity. Since we needed to perform the “mismatched” Sharpless epoxidation for the natural product, we tried to enhance the stereoselectivity by using the diisopropyltartrate instead of diethyltartrate at −20° C.³⁶ However, the stereoselectivity for the formation of epoxide (+)-4b could not be raised beyond a diastereomeric ratio of 90:10.

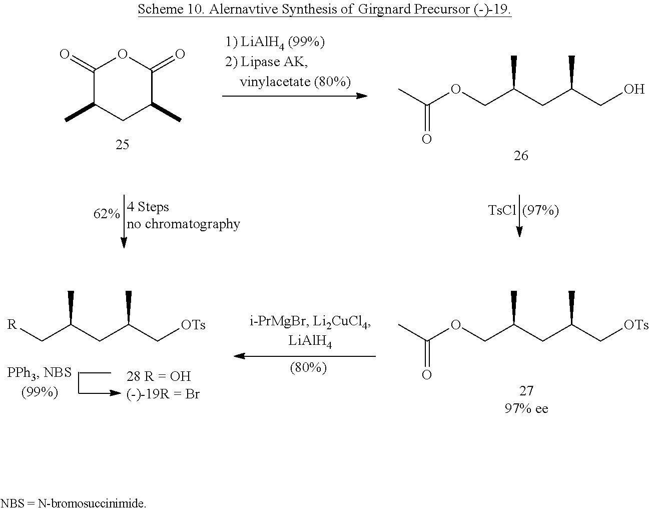

Subsequently, the oxidation level of Vittatalactone was adjusted employing the selective two step oxidation protocol developed in the first generation approach, to give the hydroxy acid 29 without any trace of over-oxidation to the β-keto-acid. Final ring closure to the natural product was accomplished with tosyl chloride in pyridine to furnish natural Vittatalactone ((+)-1b) in good yield.

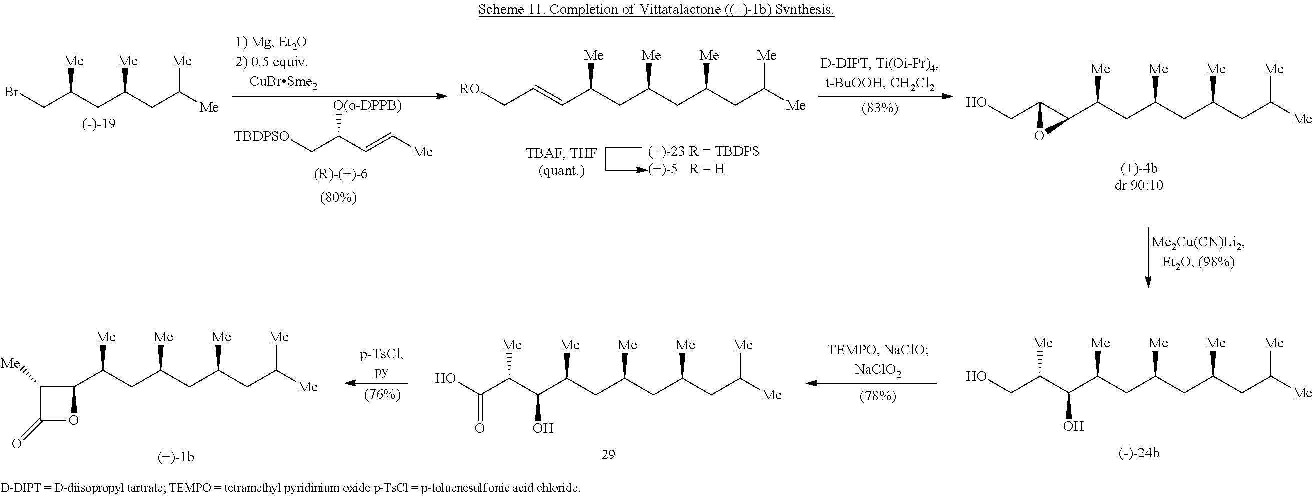

Conclusion

Through total synthesis and comparison of NMR-data of synthetic and natural material we could elucidate the absolute configuration of Vittatalactone ((+)-1b) from Acalymma vittatum. With the development of a more convergent and practical second generation synthesis we could prepare over 50 mg of the natural stereoisomer in 11 steps for the longest linear sequence and a total yield of 18%.

The successful enantioselective synthesis of two unnatural diastereomers 1a and (−)-1b as well as the natural isomer (+)-1b highlights the synthetic power of the directed allylic substitution for deoxypropionate and propionate construction.

Now, the door is open for extensive biological studies to see whether this pheromone can indeed become an environmentally friendly and efficient tool for a cucurbit plant protection strategy in the future.

Experimental Section

General Information. All reagents were commercially available unless otherwise noted. All reactions were carried out under argon (5.0) atmosphere in dried glassware. Air and moisture sensitive liquids and solutions were transferred via syringe. All solvents were dried and distilled by standard procedures. Solutions were concentrated under reduced pressure by rotary evaporation. Chromatographic purification of products was accomplished on Merck silica gel Si 60® (200-400 mesh). Melting points were measured using open glass capillaries, the values are uncorrected. Optical rotations were measured in 1.0 dm, 1.0 ml cells. The concentration in g/100 mL and the solvents are given in parentheses. Nuclear magnetic resonance spectra were acquired at 400.132 MHz and 101.626 MHz for ¹H and ¹³C, respectively and were referenced according to internal TMS standard. Data for ¹H-NMR are reported as follows: chemical shift (δ in ppm), multiplicity (s, singlet; br s, broad singlet; d, doublet; t, triplet; q, quartet; m, multiplet; m_{c}, centered multiplet), coupling constant (Hz), integration. Data for ¹³C-NMR are reported in terms of chemical shift (δ in ppm) and multiplicity. Peak assignment of hydrogen and carbon atoms was confirmed using DQF-COSY measurements and edHSQC measurements, respectively.

(R,E)-5,7-dimethyloct-3-ene. (R,E)-hex-4-en-3-yl 2-(diphenylphosphino)benzoate (8) (402 mg, 1.02 mol, 1.00 equiv., 98% ee) and copper(I)bromide dimethyl sulfide complex (105 mg, 0.510 mmol, 0.500 equiv.) were suspended in diethyl ether (20 ml). A solution of isopropyl magnesium bromide in diethyl ether (about 1.0 M, 3.6 ml, 1.2 equiv.) was added over 1.5 h at room temperature with a syringe pump. The reaction mixture was stirred for another 1 h until full conversion was observed by TLC. The reaction mixture was directly transferred on a silica gel plug and rinsed with pentane. The resulting solution was washed with aqueous sodium hydroxide (0.25 M, 50 ml) and washed with saturated aqueous sodium hydrogen carbonate (20 ml). Due to the high volatility of the product it was used in the next step without further concentration or purification. R_{f}=0.82 (pentane).

(R)-2,4-dimethylpentan-1-ol. The solution of (R,E)-5,7-dimethyloct-3-ene in diethyl ether/pentane (about 100 ml) was cooled to −78° C. Then ozone was bubbled through the solution until a blue color persisted. Immediately afterwards nitrogen was purged through the solution until the blue color disappeared. Remaining at −78° C., sodium borohydrate (1.1 g, 30 mmol, 30 equiv.) was added in portions. The reaction mixture was allowed to warm to room temperature overnight. The suspension was quenched with water (30 ml) and stirred vigorously for 2 h. The phases were separated and the aqueous phase was extracted with diethyl ether (4×30 ml). The combined organic phases were dried over sodium sulfate and concentrated in vacuo. Purification by flash chromatography (pentane/diethyl ether 1:1, Ø2 cm, length 16 cm, fraction size 6 ml, fractions 12-18) to obtain the title compound as a volatile colorless liquid (89 mg, 76% over two steps from 8). R_{f}=0.63 (diethyl ether); [α]_{D}²⁴=+10.6° (c=0.7, CHCl₃, 98% ee); to determine the ee the alcohol was converted into the corresponding benzoyl ester ((R)-2,4-dimethylpentyl benzoate): HPLC Diacel Chiralpak® AD-3 column, heptane/isopropanol 400:1, flow rate=1.0 ml/min, 20° C., detection at 227 nm, t₁=4.05 min (major), t₂=4.72 min (minor). The NMR-spectroscopic analysis is consistent with literature data.⁷

(R)-2,4-dimethylpentan-1-ol. Lithium aluminum hydride (33 mg, 0.86 mmol, 2.0 equiv.) was suspended in diethyl ether (5 ml) and cooled to −20° C. Then a solution of (R)-tert-butyl 2,4-dimethylpentanoate (80 mg, 0.43 mmol, 1.00 equiv.) in diethyl ether (5 ml) was added during 10 min. The reaction mixture was allowed to warm to room temperature overnight. It was cooled back to 0° C., quenched with saturated aqueous sodium sulfate (0.4 ml), and dried over sodium sulfate. Purification by flash chromatography (pentane/diethyl ether 1:1, Ø2 cm, length 16 cm, fraction size 7 ml, fractions 9-15) to obtain the title compound as a colorless liquid (48 mg, 96%). The NMR-spectroscopic analysis is consistent with literature data.⁷

(R)-1-bromo-2,4-dimethylpentane (14). A solution of (R)-2,4-dimethylpentan-1-ol (10 mg, 86 μmol, 1.00 equiv.) in dichloromethane (1 ml) was cooled to 0° C. Triphenyl-phosphine (33 mg, 130 μmol, 1.50 equiv.) was added and the mixture was stirred until it turned into a homogenous clear solution. N-bromosuccinimide (23 mg, 130 mol, 1.50 equiv.) was added at 0° C. The reaction mixture was allowed to warm to room temperature overnight. The dark reaction mixture was directly purified by filtration through a pad of silica gel (Ø1 cm, length 2 cm) and rinsed with pentane. The title compound was obtained as a colorless liquid (13.8 mg, 90%, contained pentane). R_{f}=0.78 (pentane); [α]_{D}²⁴=+6.2° (c=0.98, CHCl₃, 98% ee); ¹H-NMR (400 MHz, CDCl₃, TMS as internal standard): δ (ppm)=0.90 (d, J=6.4 Hz, 3H), 0.92 (d, J=6.6 Hz, 3H), 1.02 (d, J=6.6 Hz, 3H), 1.12 (ddd, J=13.6, 8.2, 6.4 Hz, 1H), 1.22-1.38 (m, 1H), 1.67 (dqqd, J=7.6, 6.6, 6.6, 6.5 Hz, 1H), 1.89 (m, 1H), 3.32 (dd, J=9.7, 6.3 Hz, 1H), 3.42 (dd, J=9.7 Hz, 4.6 Hz, 1H); ¹³C-NMR (100 MHz, CDCl₃): δ (ppm)=19.0, 23.2, 25.4, 27.0, 33.0, 42.0, 44.4. The spectroscopic data match with the reported.³⁷

(2R,4S)-2,4-dimethylpentane-1,5-diol. A suspension of (3R,5S)-3,5-dimethyldihydro-2H-pyran-2,6(3H)-dione (25) (100 mmol, 14.3 g, 1.00 equiv.) in diethyl ether (300 ml) was cooled to 0° C. Lithium aluminium hydride (200 mmol, 7.59 g, 2.00 equiv.) was added in portions under vigorous stirring over 1 h. The suspension was allowed to warm to room temperature overnight. The reaction mixture was cooled again to 0° C. and water (8 ml), aqueous sodium hydroxide solution (15%, 8 ml), diethyl ether (100 ml), and water (24 ml) were added successively. The reaction mixture was stirred until it turned from grey to white (about 2 h) and dried over sodium sulfate. The solvent was removed in vacuo to obtain the title compound as a colorless, viscous, and hygroscopic oil (13.0 g, 98%). R_{f}=0.28 (ethyl acetate); ¹H-NMR (400 MHz, CDCl₃, TMS as internal standard): δ (ppm)=0.94 (ddd, J=14.2, 7.1, 7.1 Hz, 1H) 0.95 (d, J=6.7 Hz, 6H), 1.53 (ddd, J=13.7, 6.8, 6.8 Hz, 1H), 1.74 (ddqdd, J=7.0, 6.8, 6.7, 5.7, 5.7, 2H), 1.87 (br s, 2H), 3.48 (d, J=5.7 Hz, 4H); ¹³C-NMR (100 MHz, CDCl₃): δ (ppm)=17.6 (2C), 33.1 (2C), 37.0, 67.9 (2C). The spectroscopic data match with the reported.³⁸

(2S,4R)-5-hydroxy-2,4-dimethylpentyl acetate (26). A solution of (2R,4S)-2,4-dimethyl-pentane-1,5-diol (2.00 g, 15.1 mmol, 1.00 equiv.) in tetrahydrofuran (20 ml) was cooled to 0° C. At this temperature Amano Lipase AK (110 mg) and vinyl acetate (2.10 ml, 1.95 g, 22.7 mmol, 1.50 equiv.) were added. The reaction mixture was stirred for 30 min at 0° C. and 7 h at 5° C. The enzyme was removed by suction filtration through Celite and washed with diethyl ether (40 ml). The homogeneous filtrate was concentrated in vacuo. This crude product was either directly used in the next reaction or for analytical purposes purified by flash chromatography (diethyl ether, Ø5 cm, length 16 cm, fraction size 40 ml, fractions 9-16) to obtain the clean title compound as a colorless oil (2.09 g, 79%). R_{f}=0.42 (cyclohexane/ethyl acetate 1:1); [α]_{D}²⁴=+11.42° (c=1.05, CHCl₃); ¹H-NMR (400 MHz, CDCl₃, TMS as internal standard): δ (ppm)=0.95 (d, J=6.7 Hz, 3H), 0.96 (d, J=6.7 Hz, 3H), 1.00 (ddd, J=13.8, 7.7, 7.1 Hz, 1H), 1.45 (ddd, J=13.7, 7.1, 6.6 Hz, 1H), 1.52 (br s, 1H), 1.74 (ddqdd, J=7.7, 7.1, 6.7, 6.6, 5.5 Hz, 1H), 1.90 (ddqdd, J=7.1, 6.8, 6.7, 6.6, 5.4 Hz, 1H), 2.06 (s, 3H), 3.41 (dd, J=10.3, 6.6, 1H), 3.50 (dd, J=10.3, 6.6, 1H), 3.85 (dd, J=10.8, 6.8, Hz, 1H), 3.97 (dd, J=10.8, 5.4 Hz, 1H); ¹³C-NMR (100 MHz, CDCl₃): δ (ppm)=17.2, 17.8, 20.9, 30.0, 33.0, 37.3, 68.0, 69.2, 171.3. The spectroscopic data match with the reported.³⁹

(2S,4R)-2,4-dimethyl-5-(tosyloxy)pentyl acetate (27). A solution of (2S,4R)-5-hydroxy-2,4-dimethylpentyl acetate (26) (1.70 g, 9.76 mmol, 1.00 equiv.) in dry pyridine (30 ml) was cooled to 0° C. Then p-toluenesulfonyl chloride (2.79 g, 14.6 mmol, 1.50 equiv.) was added stepwise. The reaction mixture was allowed to warm to room temperature overnight. It was quenched by the addition of water (50 ml), followed by extraction with diethyl ether (3×40 ml). The organic layers were combined and washed with saturated aqueous copper(II)sulfate solution (2×60 ml), saturated aqueous sodium hydrogen carbonate (30 ml), and brine (30 ml). The solution was dried over sodium sulfate and concentrated in vacuo. This crude product was either directly used in the next step or for analytical purposes purified by flash chromatography (cyclohexane/ethyl acetate 1:1, Ø5 cm, length 18 cm, fraction size 40 ml, fractions 7-10) to obtain the title compound as a colorless oil (3.10 g, 97%, 97% ee). R_{f}=0.52 (cyclohexane/ethyl acetate 1:1); [α]_{D}²⁴=+1.61° (c=1.24, CHCl₃; 97% ee); HPLC Chiralcel® OJ-H column, heptane/ethanol 75:25, flow rate=0.8 ml/min, 23° C., detection at 230 nm, t₁=15.37 min (major), t₂=20.83 min (minor); ¹H-NMR (400 MHz, CDCl₃, TMS as internal standard): δ (ppm)=0.90 (d, J=6.7 Hz, 3H), 0.92 (d, J=6.7 Hz, 3H), 1.00 (ddd, J=13.9, 7.4, 7.4 Hz, 1H), 1.39 (ddd, J=13.8, 6.9, 6.9, Hz, 1H), 1.79 (dqddd, J=7.4, 6.8, 6.8, 6.8, 6.8 Hz, 1H), 1.89 (qdddd, J=6.7, 6.7, 6.7, 6.2, 5.4 Hz, 1H), 2.04 (s, 3H), 2.45 (s, 3H), 3.79 (dd, J=10.8, 6.5 Hz, 1H), 3.80 (dd, J=9.5, 6.2 Hz, 1H), 3.87 (dd, J=10.7, 5.4 Hz, 1H), 3.88 (dd, J=10.6, 5.3 Hz, 1H), 7.33-7.37 (m, 2H), 7.76-7.81 (m, 2H); ¹³C-NMR (100 MHz, CDCl₃): δ (ppm)=17.1, 17.4, 20.9, 21.6, 29.8, 30.3, 36.9, 68.8, 74.7, 127.8 (2C), 129.8 (2C), 133.1, 144.7, 171.1. The spectroscopic data match with the reported.⁴⁰

(2S,4S)-2,4,6-trimethylheptan-1-ol (28). Lithium chloride (42 mg, 1.0 mmol, 0.60 equiv.) and copper(II)chloride (68 mg, 0.50 mmol, 0.30 equiv.) were flame dried in a flask and dissolved in tetrahydrofuran (10 ml) to obtain an orange solution of lithium tetrachlorocuprate(II) (0.1 M in tetrahydrofuran).

A solution of (2S,4R)-2,4-dimethyl-5-(tosyloxy)pentyl acetate (27) (1.01 g, 3.07 mmol, 1.00 equiv.) in tetrahydrofuran (20 ml) was cooled to −78° C. The lithium tetrachlorocuprate(II) solution (0.1 M in tetrahydrofuran, 10 ml, 1.0 mmol, 0.30 equiv.) was added via syringe. After 10 min an isopropylmagnesium bromide solution (1.0 M in tetrahydrofuran, 15.4 ml, 15.4 mmol, 5.00 equiv.) was added via cannula. The reaction mixture was allowed to warm to room temperature overnight. The reaction mixture was then cooled to 0° C. and lithium aluminum hydride (117 mg, 3.07 mmol, 1.00 equiv.) was added. It was again allowed to warm to room temperature overnight. The reaction mixture was cooled to 0° C. and quenched by the addition of saturated ammonium chloride solution (50 ml). Tetrahydrofuran was removed in vacuo. The residue was taken up in diethyl ether (50 ml). The phases were separated and the aqueous phase was extracted with diethyl ether (4×50 ml). If no good phase separation was observed some sodium potassium tartrate was added. The combined organic phases were washed with brine (30 ml), dried over sodium sulfate, and concentrated in vacuo. Purification by flash chromatography (petroleum ether/diethyl ether 1:1, Ø4 cm, length 20 cm, fraction size 25 ml, fractions 22-28) or short path distillation (10 mbar, 120-130° C. oil bath temperature) to obtain the title compound as a colorless oil (88 mg, 80%). R_{f}=0.18 (cyclohexane/ethyl acetate 9:1); [α]_{D}²⁴=−16.3° (c=1.03, CHCl₃); ¹H-NMR (400 MHz, CDCl₃, TMS as internal standard): δ (ppm)=0.84 (d, J=6.5 Hz, 3H), 0.86 (d, J=6.6 Hz, 3H), 0.87 (d, J=6.6 Hz, 3H), 0.92 (d, J=6.7 Hz, 3H), 0.89-0.99 (m, 2H), 1.11 (ddd, J=13.7, 8.9, 5.0 Hz, 1H), 1.27 (ddd, J=13.6, 6.8, 6.8 Hz, 1H), 1.49 (br s, 1H), 1.50-1.79 (m, 3H), 3.37 (dd, J=10.5, 6.8 Hz, 1H), 3.53 (dd, J=10.5, 5.2 Hz, 1H); ¹³C-NMR (100 MHz, CDCl₃): δ (ppm)=17.3, 20.5, 22.0, 23.7, 25.2, 27.7, 33.0, 41.6, 46.5, 68.4. The spectroscopic data match with the reported.⁴¹

(2S,4S)-1-bromo-2,4,6-trimethylheptane ((−)-19). A solution of (2S,4S)-2,4,6-trimethylheptan-1-ol (28) (800 mg, 5.05 mmol, 1.00 equiv.) in dichloromethane (10 ml) was cooled to 0° C. Triphenylphosphine (1.99 g, 7.60 mmol, 1.50 equiv.) was added at once and the mixture was stirred until it became a homogenous solution. N-bromosuccinimide (1.35 g, 7.60 mmol, 1.50 equiv.) was added in portions over 15 min at 0° C. The reaction mixture was allowed to warm to room temperature overnight. The dark reaction mixture was concentrated in vacuo, purified by filtration through a pad of silica gel (Ø5 cm, length 10 cm), and rinsed with pentane. The title compound was obtained as a colorless liquid (1.06 g, 96%) by removal of the solvent. R_{f}=0.78 (pentane); [α]_{D}²⁴=−2.16° (c=1.25, CHCl₃); ¹H-NMR (400 MHz, CDCl₃, TMS as internal standard): δ (ppm)=0.85 (d, J=6.6 Hz, 3H), 0.85 (d, J=6.6 Hz, 3H), 0.88 (d, J=6.6 Hz, 3H), 0.93-1.13 (m, 3H), 1.01 (d, J=6.6 Hz, 3H), 1.37 (ddd, J=13.6, 6.9, 6.7 Hz, 1H), 1.48-1.61 (m, 1H), 1.60-1.71 (m, 1H), 1.83-1.96 (m, 1H), 3.31 (dd, J=9.8, 6.3 Hz, 1H), 3.41 (dd, J=9.8, 6.3 Hz, 1H); ¹³C-NMR (100 MHz, CDCl₃): δ (ppm)=19.5, 20.2, 22.2, 23.5, 25.2, 27.7, 32.4, 41.7, 43.0, 46.7. The spectroscopic data match with the reported data of the enantiomeric compound.⁴²

tert-butyldiphenyl((4S,6S,8S,E)-4,6,8,10-tetramethylundec-2-enyloxy)silane ((+)-23). Magnesium powder (330 mg, 14 mmol, 5.0 equiv.) was suspended in diethyl ether (5 ml) and etched with dibromoethane (0.1 ml). Then the solvent was removed via syringe from the suspension. The etching process was repeated three times. Diethyl ether (5 ml) and one drop of dibromoethane were added (about 20 μl) to the remaining magnesium powder. Then (2S,4S)-1-bromo-2,4,6-trimethylheptane ((−)-19) (600 mg, 2.71 mmol, 1.20 equiv.) was added over 20 min and stirred for 30 min at room temperature. A syringe was charged with the Grignard suspension.

A separate flask was charged with copper(I)bromide dimethyl sulfide complex (232 mg, 1.13 mmol, 0.500 equiv.), (R,E)-1-(tert-butyldiphenylsilyloxy)pent-3-en-2-yl 2-(diphenyl-phosphino)benzoate ((R)-(+)-6) (1.42 g, 2.26 mmol, 1.00 equiv.), and diethyl ether (50 ml). The Grignard suspension was added via a syringe pump over 1.25 h and stirred for another 1.5 h. The reaction mixture was concentrated in vacuo. Purification by flash chromatography (petroleum ether, Ø5 cm, length 19 cm, fraction size 45 ml, fractions 6-12) to obtain the title compound as a colorless oil (0.99 g, 80%, dr >95:5 by NMR). R_{f}=0.25 (petroleum ether); [α]_{D}²⁴=+3.00° (c=1.10, CHCl₃); ¹H-NMR (400 MHz, CDCl₃, TMS as internal standard): δ (ppm)=0.79 (d, J=6.5 Hz, 3H), 0.83 (d, J=6.5 Hz, 3H), 0.85 (d, J=6.5 Hz, 6H), 0.96 (d, J=6.7 Hz, 3H), 0.81-0.98 (m, 3H), 1.05 (s, 9H), 1.08-1.31 (m, 3H), 1.42-1.72 (m, 3H), 2.17-2.29 (m, 1H), 4.15-4.17 (m, 2H), 5.44-5.56 (m, 2H), 7.34-7.44 (m, 6H), 7.65-7.71 (m, 4H); ¹³C-NMR (100 MHz, CDCl₃): δ (ppm)=19.2, 20.2, 20.3, 21.7, 22.1, 23.6, 25.2, 26.9 (3C), 27.4, 27.5, 33.9, 44.4, 46.1, 46.8, 64.7, 127.1, 127.6 (2C), 129.5 (4C), 134.0 (2C), 135.6 (4C), 136.9. The spectroscopic data match with the reported data of the enantiomeric compound.²⁵

(4S,6S,8S,E)-4,6,8,10-tetramethylundec-2-en-1-ol ((+)-5). To a solution of tert-butyldiphenyl(4S,6S,8S,E)-4,6,8,10-tetramethylundec-2-enyloxy)silane ((+)-23) (200 mg, 430 μmol, 1.00 equiv.) in tetrahydrofuran (3 ml) was added tetrabutylammonium fluoride trihydrate (407 mg, 1.30 mmol, 3.00 equiv.) at room temperature. After 4.5 h the TLC showed complete conversion. The reaction mixture was concentrated in vacuo. Purification by flash chromatography (petroleum ether/tert-butyl methyl ether, Ø3 cm, length 17 cm, fraction size 25 ml, fractions 21-27) to obtain the title compound as a colorless oil (96 mg, 98%). R_{f}=0.15 (petroleum ether/ethyl acetate 9:1); [α]_{D}²⁴=+9.89° (c=2.81, CHCl₃); ¹H-NMR (400 MHz, CDCl₃, TMS as internal standard): δ (ppm)=0.80 (d, J=6.5 Hz, 3H), 0.83 (d, J=6.5 Hz, 3H), 0.84 (d, J=6.6 Hz, 3H), 0.87 (d, J=6.6 Hz, 3H), 0.98 (d, J=6.69 Hz, 3H), 0.85-1.01 (m, 3H), 1.04-1.19 (m, 2H), 1.28 (ddd, J=13.9, 9.5, 4.6 Hz, 1H), 1.34 (br s, 1H), 1.46-1.59 (m, 2H), 1.60-1.69 (m, 1H), 2.20-2.32 (m, 1H), 4.08-4.11 (m, 2H), 5.51 (dddd, J=15.4, 7.9, 1.1, 1.1 Hz, 1H), 5.61 (dddd, J=15.4, 5.6, 5.6, 0.6 Hz, 1H); ¹³C-NMR (100 MHz, CDCl₃): δ (ppm)=20.3, 20.3, 21.5, 22.1, 23.6, 25.2, 27.5, 27.5, 34.0, 44.3, 46.0, 46.8, 63.9, 127.3, 139.1. The spectroscopic data match with the reported data of the enantiomeric compound.²⁵

((2R,3R)-3-(2S,4S,6S)-4,6,8-trimethylnonan-2-yl)oxiran-2-yl)methanol ((+)-4b). tert-Butyl hydroperoxide (about 4 M in decane, 0.25 ml, about 4 equiv.) was diluted with dichloromethane (0.75 ml) and dried over 4 Å ground molecular sieves for 2 h. A Schlenk flask was charged with titanium tetraisopropoxide (20 μl, 68 μmol, 0.25 equiv.) and dichloromethane (2 ml) and dried over 4 Å ground molecular sieves for 1.5 h. (4S,6S,8S,E)-4,6,8,10-tetramethylundec-2-en-1-ol ((+)-5) (60.0 mg, 0.275 mmol, 1.00 equiv.) was dissolved in dichloromethane (1 ml) and dried over 4 Å ground molecular sieves for 30 min. The titanium tetraisopropoxide was cooled to −20° C. and stirred for another 30 min. Then D-(−)-diisopropyltartrate (17 μl, 81 μmol, 0.30 equiv.) was added. After 30 min the solution of the allyl alcohol was added slowly over 10 min. Directly afterwards the tert-butyl hydroperoxide solution was added and the reaction mixture stirred overnight at −20° C. (about 20 h, TLC shows full conversion).

The reaction mixture was quenched at −20° C. by addition of a solution containing iron(II)sulfate heptahydrate (3.3 g), citric acid (1.1 g), and water (10 ml). The mixture was stirred vigorously and allowed to warm to room temperature for 30 min, followed by extraction with diethyl ether (3×8 ml) and drying over sodium sulfate. Purification by flash chromatography (petroleum ether/diethyl ether 8:2, Ø2 cm, length 20 cm, fraction size 10 ml, fractions 26-31) to obtain the title compound as a colorless oil (54 mg, 83%, dr 90:10 determined by ¹H-NMR). R_{f}=0.18 (cyclohexane/ethyl acetate 8:2); [α]_{D}²⁴=+16.27° (c=8.05, CHCl₃); ¹H-NMR (400 MHz, CDCl₃, TMS as internal standard): δ (ppm)=0.82 (d, J=6.5 Hz, 3H), 0.84 (d, J=6.5 Hz, 3H), 0.86 (d, J=6.5 Hz, 3H), 0.87 (d, J=6.6 Hz, 3H), 0.84-0.97 (m, 2H), 1.02 (d, J=6.6 Hz, 3H), 1.02-1.18 (m, 3H), 1.36 (ddd, J=13.7, 8.6, 5.2 Hz, 1H), 1.44-1.51 (m, 1H), 1.51-1.69 (m, 3H), 1.72 (br s, 1H), 2.68 (dd, J=7.7, 2.4 Hz, 1H), 2.99 (ddd, J=4.5, 2.5, 2.5 Hz, 1H), 3.61 (dd, J=12.5, 3.7 Hz, 1H), 3.92 (dd, J=12.5, 2.1 Hz, 1H); ¹³C-NMR (100 MHz, CDCl₃): δ (ppm)=18.0, 20.4, 20.6, 22.0, 23.7, 25.2, 27.4, 27.5, 32.9, 41.5, 45.8, 46.6, 58.5, 60.6, 61.8. The spectroscopic data match with the reported data of the enantiomeric compound.²⁵

(2S,3R,4S,6S,8S)-2,4,6,8,10-pentamethylundecane-1,3-diol ((−)-24b). Copper(I)cyanide (223 mg, 2.49 mmol, 3.00 equiv.) was suspended in diethyl ether (10 ml) and cooled to −78° C. A solution of methyl lithium (about 1.5 M in diethyl ether, 5 ml, 6 equiv.) was slowly added. The solution was stirred for 20 min at −78° C. Then ((2R,3R)-3-((2S,4S,6S)-4,6,8-trimethylnonan-2-yl)oxiran-2-yl)methanol ((+)-4b) (dr 90:10, 201 mg, 0.829 mmol, 1.00 equiv.) dissolved in diethyl ether (5 ml) was added slowly. The solution was stirred for 1 h at −78° C. and allowed to warm to room temperature overnight. The reaction was cooled down to 0° C., quenched with saturated ammonium chloride solution (20 ml), and stirred vigorously for 10 min. Then aqueous ammonia solution (about 25%, 5 ml) was added and the mixture was stirred until the aqueous phase turned into a light blue (about 5-20 min). The phases were separated and the aqueous phase was extracted with ethyl acetate (4×25 ml). The combined organic phases were dried over sodium sulfate and concentrated in vacuo. Purification by flash chromatography (petroleum ether/diethyl ether 1:1, Ø4 cm, length 18 cm, fraction size 30 ml, fractions 30-42) to obtain the title compound as a colorless oil (209 mg, 97%, major diastereomer 189 mg). R_{f}=0.52 (diethyl ether, major diasteromer); R_{f}=0.47 (diethyl ether, minor diasteromer); [α]_{D}²⁴=−4.48° (c=0.87, CHCl₃); ¹H-NMR (400 MHz, CDCl₃, TMS as internal standard): δ (ppm)=0.82 (d, J=6.9 Hz, 3H), 0.84 (d, J=6.7 Hz, 3H), 0.84 (d, J=6.5 Hz, 3H), 0.85 (d, J=6.8 Hz, 3H), 0.86 (d, J=6.8 Hz, 3H), 0.88 (d, J=6.6 Hz, 3H), 0.84-0.96 (m, 2H), 0.99 (ddd, J=13.6, 8.0, 6.8 Hz, 1H), 1.11 (ddd, J=13.7, 9.0, 4.9 Hz, 1H), 1.18 (ddd, J=13.5, 7.2, 6.3 Hz, 1H), 1.37 (ddd, J=13.7, 7.8, 6.0 Hz, 1H), 1.52-1.70 (m, 3H), 1.73-1.84 (m, 1H), 1.83-1.95 (m, 1H), 2.38 (br s, 2H), 3.47 (dd, J=9.1, 2.5 Hz, 1H), 3.67 (dd, J=10.7, 7.8 Hz, 1H), 3.73 (dd, J=10.7, 3.8 Hz, 1H); ¹³C-NMR (100 MHz, CDCl₃): δ (ppm)=12.9, 13.5, 20.5, 20.6, 22.0, 23.8, 25.2, 27.0, 27.6, 32.0, 37.5, 41.4, 45.9, 46.5, 69.0, 79.5. The spectroscopic data match with the reported data of the enantiomeric compound.¹⁰

(2R,3R,4S,6S,8S)-3-hydroxy-2,4,6,8,10-pentamethylundecanoic acid (29). To a solution of (2S,3R,4S,6S,8S)-2,4,6,8,10-pentamethylundecane-1,3-diol ((−)-24b) (8.0 mg, 31 mmol, 1.0 equiv.) in dichloromethane (4 ml) was added saturated sodium hydrogen carbonate solution (20 ml) and sodium bromide (about 4 mg). The suspension was cooled to 0° C. At this temperature 2,2,6,6-tetramethylpiperidine-1-oxyl free radical (about 4 mg) and after 5 min aqueous sodium hypochlorite (0.01 M, 4.0 ml) were added. After stirring for 25 min at 0° C. TLC showed complete conversion. At 0° C. the reaction was quenched with saturated sodium thiosulfate solution (25 ml) and allowed to warm to room temperature. The reaction mixture was extracted with ethyl acetate (5×8 ml), dried over sodium sulfate, and concentrated in vacuo. The crude aldehyde ((2R,3R,4S,6S,8S)-3-hydroxy-2,4,6,8,10-pentamethylundecanal) (R_{f}=0.79 (diethyl ether)) was used in the next step without any further purification.

Preparation of the stock solution: tert-butanol (1.6 ml), 2-methyl-2-butene (purity 80%, 0.7 ml), sodium chlorite (28 mg), sodium dihydrogen phosphate (23 mg), and water (1.2 ml) were stirred vigorously in a small test tube for 10 min.

The freshly prepared stock solution was added to the aldehyde and stirred at room temperature for 3.5 h. Subsequently, the reaction mixture was diluted with water (8 ml) and extracted with ethyl acetate (5×8 ml). Purification by flash chromatography (cyclohexane/ethyl acetate 1:1 with 1% acidic acid, Ø1 cm, length 18 cm, fraction size 4 ml, fractions 21-27) to obtain the title compound as a viscous oil (6.5 mg, 78%). R_{f}=0.26 (ethyl acetate); [α]_{D}²⁴=−8.00° (c=0.85, CHCl₃); ¹H-NMR (400 MHz, CDCl₃, TMS as internal standard): δ (ppm)=0.83 (d, J=6.5 Hz, 3H), 0.84 (d, J=6.5 Hz, 3H), 0.86 (d, J=6.5 Hz, 3H), 0.88 (d, J=6.7 Hz, 6H), 0.88-0.96 (m, 2H), 1.00 (ddd, J=13.7, 8.0, 6.8 Hz, 1H), 1.10 (ddd, J=13.7, 9.0, 4.9 Hz, 1H), 1.13-1.19 (m, 1H), 1.19 (d, J=7.2 Hz, 3 H), 1.45 (ddd, J=13.7, 7.6, 6.1 Hz, 1H), 1.52-1.70 (m, 3H), 1.77 (qdddd, J=6.7, 6.5, 6.5, 6.5, 3.0 Hz, 1H), 2.67 (dq, J=8.6, 7.1 Hz, 1H), 3.66 (dd, J=8.7, 3.0 Hz, 1H); ¹³C-NMR (100 MHz, CDCl₃): δ (ppm)=13.0, 14.0, 20.5, 20.6, 22.0, 23.8, 25.2, 27.0, 27.6, 31.5, 41.3, 43.3, 45.8, 46.5, 74.9, 181.0. The spectroscopic data match with the reported data of the enantiomeric compound.²⁵

(+)-Vittatalactone ((+)-1b). A solution of (2R,3R,4S,6S,8S)-3-hydroxy-2,4,6,8,10-pentamethylundecanoic acid (29) (39.9 mg, 0.146 mmol, 1.00 equiv.) in dry pyridine (0.4 ml) was cooled to 0° C. p-Toluenesulfonyl chloride (57 mg, 0.29 mmol, 2.0 equiv.) was added at once. The solution was allowed to warm to room temperature and stirred overnight. After TLC showed complete consumption of starting material (about 12 h) the reaction mixture was diluted with diethyl ether (10 ml) and water (10 ml). The phases were separated and the aqueous phase was extracted with diethyl ether (3×10 ml). The combined organic phases were washed with saturated aqueous sodium hydrogen carbonate (5 ml), dried over sodium sulfate, and concentrated in vacuo (200 mbar, 40° C.). Purification by flash chromatography (pentane/diethyl ether 9:1, Ø2 cm, length 16 cm, fraction size 5 ml, fractions 10-18) to obtain the title compound as a volatile colorless liquid (28.3 mg, 76%). R_{f}=0.33 (pentane/diethyl ether 9:1); [α]_{D}²⁴=+1.2° (c=1.29, CH₂Cl₂); ¹H-NMR (400 MHz, CDCl₃, TMS as internal standard): δ (ppm)=0.84 (d, J=6.6 Hz, 6H), 0.88 (d, J=6.6 Hz, 3H), 0.90 (d, J=6.6 Hz, 3H), 0.86-0.94 (m, 2H), 1.02 (d, J=6.6 Hz, 3H), 0.97-1.05 (m, 1H), 1.10 (ddd, J=13.6, 9.3, 4.5 Hz, 1H), 1.16-1.34 (m, 2H), 1.39 (d, J=7.5 Hz, 3H), 1.50-1.70 (m, 3H), 1.87 (ddqd, J=8.4, 8.4, 6.5, 5.0 Hz, 1H), 3.25 (qd, J=7.5, 4.1 Hz, 1H), 3.87 (dd, J=8.2, 4.1 Hz, 1H); ¹³C-NMR (100 MHz, CDCl₃): δ (ppm)=12.9, 15.8, 20.8, 21.0, 21.8, 23.9, 25.2, 27.3, 27.7, 34.8, 39.8, 45.2, 46.0, 48.9, 83.8, 172.0. The spectroscopic data match with the reported data of the enantiomeric compound.²⁵

### REFERENCES

¹Cavanagh, A.; Hazzard, R.; Adler, L. S.; Boucher, J. J. Econ. Entomol. 2009, 102, 1101-1107.
²(a) Garcia-Salazar, C. G.; Gildow, F. E.; Fleischer, S. J.; Cox-Foster, D.; Lukezic, F. L. Environ. Entomol. 2000, 29, 542-550; (b) Garcia-Salazar, C. G.; Gildow, F. E.; Fleischer, S. J.; Cox-Foster, D.; Lukezic, F. L. Can. Entomol. 2000, 132, 1-13.
³(a) Smyth, R.; Hoffmann, M. P. Physiol. Entomol. 2002, 27, 325-242; (b) Smyth, R.; Hoffmann, M. P. J. Insect Behav. 2003, 16, 347-359.
⁴(a) Brust, G. E. Environ. Entomol. 1997, 26, 580-584; (b) Fleischer, S. J.; deMackiewicz, D.; Gildow, F. E.; Lukezic, F. L. Environ. Entomol. 1999, 28, 470-476.
⁵Morris, B. D.; Smyth, R. R.; Foster, S. P.; Hoffmann, M. P.; Roelofs, W. L.; Franke, S.; Francke, W. J. Nat. Prod. 2005, 68, 26-30.
⁶Schmidt, Y.; Breit, B. Org. Lett. 2009, 11, 4767-4769.
⁷(a) Umezawa, H.; Aoyagi, T.; Uotani, K.; Hamada, M.; Takeuchi, T.; Takahashi, S. J. Antibiot. 1980, 33, 1594-1596; (b) Uotani, K.; Naganawa, H.; Kondo, S.; Aoyagi, T.; Umezawa, H. J. Antibiot. 1982, 35, 1495-1499; (c) Nonaka, Y.; Ohtaki, H.; Ohtsuka, E.; Kocha, T.; Fukuda, T.; Takeuchi, T.; Aoyagi, T. J. Enzyme Inhib. 1995, 10, 57-63.
⁸(a) Uotani, K.; Naganawa, H.; Aoyagi, T.; Umezawa, H. J. Antibiot. 1982, 35, 1670-1674; for a thorough overview on polyketide biosynthesis, see: Hertweck, C. Angew. Chem. Int. Ed. 2009, 48, 4699-4716.
⁹By comparison of NMR data we found a strong splitting (0.2 and 0.3 ppm, respectively) for the chemical shift of the methylene protons, which has been shown to be typical for the internal methylene unit of a syn-deoxypropionate unit. In case of the anti-configured diastereomers the chemical shift differences decrease. For experimental data, see: (a) Herber, C.; Breit, B. Chem. Eur. J. 2006, 12, 6684-6691; (b) Herber, C.; Breit, B. Eur. J. Org. Chem. 2007, 3512-3519; (c) Hanessian, S.; Yang, Y.; Giroux, S.; Mascitti, V.; Ma, J.; Raeppel, F. J. Am. Chem. Soc. 2003, 125, 13784.13792; for a theoretical overview of conformation control, see: d) Hoffmann, R. W. Angew. Chem. Int. Ed. 2000, 39, 2054-2070 and cited references.
¹⁰(a) For a recent review, see: Schmidt, Y.; Breit, B. Chem. Rev. 2008, 108, 2928-2952; (b) Reiss, T.; Breit, B. Chem. Eur. 12009, 15, 6345-6348; (c) Herber, C.; Breit, B. Angew. Chem. 2004, 116, 3878-3880; Angew. Chem. Int. Ed 2004, 43, 3790-3792; (d) Herber, C.; Breit, B. Chem. Eur. 12006, 12, 6684-6691.
¹¹(a) Reiss, T.; Breit, B. Org. Lett. 2009, 11, 3286-3289; (b) Rein, C.; Demel, P.; Outten, R. A.; Netscher, T.; Breit, B. Angew. Chem. Int. Ed 2007, 46, 8670-8673; (c) Herber, C.; Breit, B. Angew. Chem. Int. Ed. 2005, 44, 5267-5269; (d) Herber, C.; Breit, B. Eur. J. Org. Chem. 2007, 3512-3519.
¹²(a) Goodhue, C. T.; Schaeffer, J. R. Biotechnol. Bioeng. 1971, 13, 203-214; (b) Ohta, H.; Tetsukawa, H.; Noto, N. J. Org. Chem. 1982, 47, 2400-2404; (c) Molinari, F.; Gandolfi, R.; Villa, R.; Urban, E.; Kiener, A. Tetrahedron: Asym. 2003, 14, 2041-2043.
¹³We received the alcohol 10 as a generous gift from Novartis; for synthesis of 10, see: Walkup, R. D.; Boatman Jr., P. D.; Kane, R. R.; Cunningham, R. T. Tetrahedron Lett., 1991, 32, 3937-3940.
¹⁴Fouquet, C.; Schlosser M. Angew. Chem. 1974, 86, 50-51; Angew. Chem. Int. Ed. Engl. 1974, 13, 82-83.
¹⁵Tamura, M.; Kochi, J. Synthesis 1971, 303-305.
¹⁶Studte, C.; Breit, B. Angew. Chem. Int. Ed. 2008, 47, 5451-5455.
¹⁷Mukaiyama, T. Angew. Chem. 1976, 88, 111-120; Angew. Chem. Int. Ed. 1976, 15, 94-103.
¹⁸(a) Rosenthaler, L. Biochem. Z 1908, 14, 238-253; (b) Becker, W.; Benthin, U.; Eschenhof, E.; Pfeil, E. Biochem. Z. 1963, 337, 156-166.
¹⁹Effenberger, F.; Gaupp, S. Tetrahedron: Asym. 1999, 10, 1765-1775.
²⁰Pinner, A.; Klein, F. Ber. Dtsch. Chem. Ges. 1878, 11, 1475-1487.
²¹Warmerdam, E. G. J. C.; van den Nieuwendijk, A. M. C. H.; Brussee, J.; Kruse, C. G.; van der Gen, A. Tetrahedron: Asym. 1996, 7, 2539-2550.
²²Höfle, G.; Steglich, W.; Vorbrüggen, H. Angew. Chem. 1978, 90, 602-615; Angew. Chem. Int. Ed. Engl. 1978, 17, 569-583.
²³(a) Hoots, J. E.; Rauchfuss, T. B.; Wrobleski, D. A. Inorganic Synthesis 1982, 21, 175-180; (b) Schmidt, Y.; Kemme, S. T.; Grünanger, C. U.; Laungani, A. C.; Breit, B. Synthesis 2010, accepted for publication; (c) o-DPPBA is commercially available on a multigram scale from SinoMax Solutions Co., Ltd., Beijing, China, http://www.sinomaxchem.com.
²⁴Effenberger, F. Angew. Chem. Int. Ed. Engl. 1994, 33, 1555-1564.
²⁵Mitsunobu, O. Synthesis 1981; 1-28.
²⁶Careful cleaning of the used Mg-powder is essential for this transformation, see also ref. 6.
²⁷Reiss, T.; Breit, B. Chem. Eur. J. 2009, 15, 6345-6348.
²⁸(a) Katsuki, T.; Sharpless, K. B. J. Am. Chem. Soc. 1980, 102, 5974-5976; (b) for a review, see: Katsuki, T. in Comprehensive Asymmetric Catalysis; Jacobsen, E. N., Pfaltz, A., Yamamoto, H., Eds.; Springer: Heidelberg, 1999, Vol. II, pp 621-677.
²⁹Marshall, J. A.; Lu, Z.-H.; Johns, B. A. J. Org. Chem. 1998, 63, 817-823.
³⁰(a) Hu, T.; Takanaka, N.; Panek, J. S. J. Am. Chem. Soc. 2002, 124, 12806-12815; (b) Anelli, P. L.; Biffi, C.; Montanari, F.; Quici, S. J. Org. Chem. 1987, 52, 2559-2562.
³¹Miyazawa, T.; Endo, T. J. Org. Chem. 1985, 50, 3930-3931; Inokuchi, T.; Matsumoto, S.; Nishiyama, T.; Torii, S. J. Org. Chem. 1990, 55, 462-466; for an overview, see: de Nooy, A. E. J.; Besemer, A. C.; van Bekkum, H. Synthesis 1996, 1153-1174.
³²Pinnick, H. W.; Balkrishna, S. B.; Childers Jr., W. E. Tetrahedron 1981, 37, 2091-2096.
³³Adam, W.; Baeza, J.; Liu, J.-C. J. Am. Chem. Soc. 1972, 94, 2000-2006.
³⁴Fujita, K.; Mori, K. Eur. J. Org. Chem. 2001, 493-502.
³⁵The enantiomeric excess was determined after tosylation to 25.
³⁶(a) Sharpless, K. B.; Behrens, C. H.; Katsuki, T.; Lee A. W. M.; Martin, V. S.; Takatani, M.; Viti, S. M.; Walker F. J.; Woodard S. S. Synthesis 1983, 589-604; (b) Gao, Y.; Hanson, R. M.; Klunder, J. M.; Ko, S. Y.; Masamune, H., Sharpless, K. B. J. Am. Chem. Soc. 1987, 109, 5765-5780.
³⁷Tang, R.; Webster, F. X.; Müller-Schwarze, D. J. Chem. Ecol. 1995, 21, 1745.
³⁸Ley, S. V.; Tackett, M. N.; Maddess, M. L.; Anderson, J. C.; Brennan, P. E.; Cappi, M. W.; Heer, J. P.; Helgen, C.; Kori, M.; Kouklovsky, C.; Marsden, S. P.; Norman, J.; Osborn, D. P.; Palomero, M. Á.; Pavey, J. B. J.; Pinel, C.; Robinson, L. A.; Schnaubelt, J.; Scott, J. S.; Spilling, C. D.; Watanabe, H.; Wesson, K. E.; Willis, M. C. Chem. Eur. 12009, 15, 2874.
³⁹Fujita, K.; Mori, K. Eur. J. Org. Chem. 2001, 493.
⁴⁰van Summeren, R. P.; Reijmer, S. J. W.; Fering a, B. L.; Minnaard, A. J. Chem. Commun. 2005, 1387.
⁴¹Stahl, M.; Schopfer, U. J. Chem. Soc., Perkin Trans. 2 1997, 905.
⁴²Schmidt, Y.; Breit, B. Org. Lett. 2009, 11, 4767.

1. A method for preparing a compound of Formula (1) 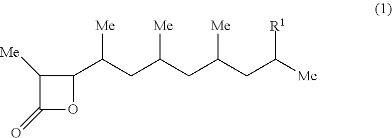
comprising the following steps:
a) reacting a compound of Formula (2) 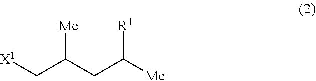
with a compound of Formula (3) 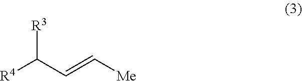
to obtain a compound of Formula (4) 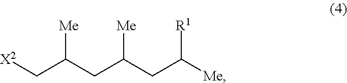
b) reacting the compound of Formula (4) with a compound of Formula (5) 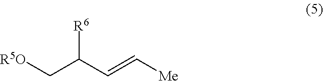
to obtain a compound of Formula (6) 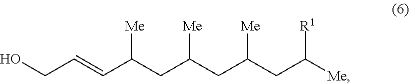
and
c) converting the compound of Formula (6) into the compound of Formula (1);
wherein
R¹ is hydrogen or methyl,
X¹ is halogen or —SR²,
R² is a hydrocarbon group,
X² is halogen or —SR²,
R³ is a leaving group,
R⁴ is a hydrocarbon group,
R⁵ is a protecting group, and
R⁶ is a leaving group.

2. The method of claim 1, wherein R¹ is methyl, and the compound of Formula (2) is prepared by converting a compound of Formula (7), 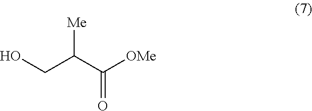
or a compound of Formula (8), 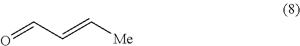
or a compound of Formula (9) 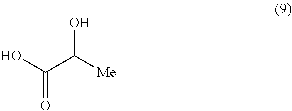
or a compound of Formula (10) 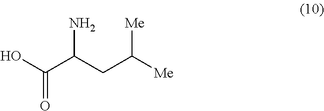
into a compound of Formula (2).

3. The method of claim 1, wherein said X¹ is Br.

4. The method of claim 1, wherein step a) further comprises the steps of:
(i) reacting a compound of Formula (2) 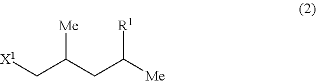
with the compound of Formula (3) to obtain a compound of Formula (11) 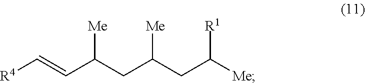
and
(ii) converting the compound of Formula (11) into the compound of Formula (4); wherein R⁴ is as defined in claim 1.

5. The method of claim 1, wherein the compound of Formula (5) is prepared by
(i) converting a compound of Formula (12) 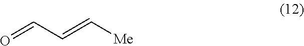
into a compound of Formula (13) 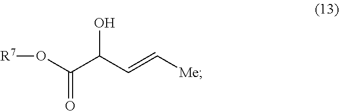
(ii) converting the compound of Formula (13) into a compound of Formula (14) 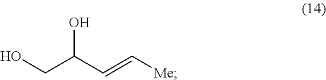
(iii) converting the compound of Formula (14) into a compound of Formula (15) 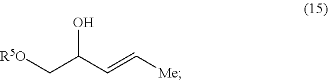
and
(iv) converting the compound of Formula (15) into the compound of Formula 5, wherein R⁵ is as defined in claim 1, and R⁷ is an alkyl group.

6. The method according to claim 1, wherein step c) further comprises the steps of:
(i) converting the compound of Formula (6) into a compound of Formula (16) 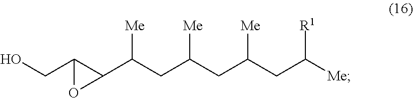
(ii) converting the compound of Formula (16) into a compound of Formula (17) 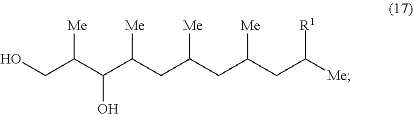
and
(iii) converting the compound of Formula (17) into the compound of Formula (1).

7. A method for preparing a compound of Formula (1) 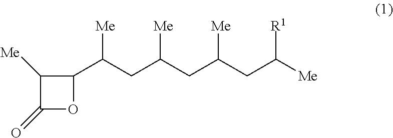
comprising the following steps:
a) converting a compound of Formula (18) 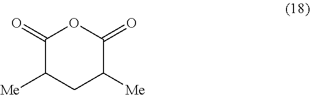
into a compound of Formula (4) 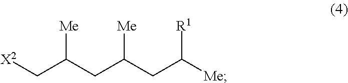
b) reacting the compound of Formula (4) with a compound of Formula (5) 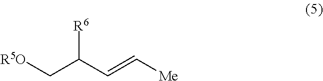
to obtain a compound of Formula (6) 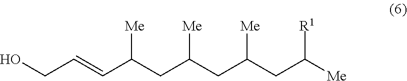
and
c) converting the compound of Formula (6) into the compound of Formula (1); wherein
R¹ is hydrogen or methyl,
X² is halogen or —SR²,
R² is hydrocarbon group;
R⁵ is a protecting group, and
R⁶ is a leaving group.

8. The method of claim 7, wherein step a) further comprises:
converting the compound of Formula (18a) 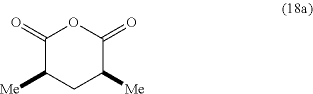
into the compound of Formula (4a) 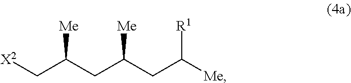
wherein R¹ and X² are as defined in claim 7.

9. The method of claim 7, wherein step a) further comprises the steps of:
(i) converting the compound of Formula (18a) into a compound of Formula (19a) 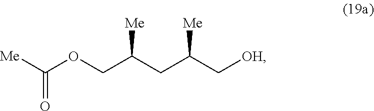
(ii) converting the compound of Formula (19a) into a compound of Formula (20a) 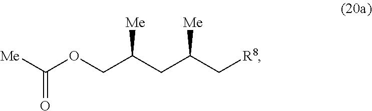
and
(iii) converting the compound of Formula (20a) into the compound of Formula (4a); wherein R⁸ is a leaving group.

10. The method of claim 8, wherein step b) further comprises reacting the compound of Formula (4a) with a compound of Formula (5a) 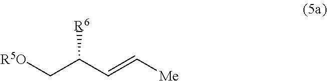
to obtain a compound of Formula (6a) 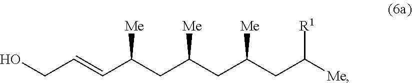
wherein R⁶ is a leaving group, and R⁵ is a protecting group.

11. The method of claim 7, wherein R¹ is methyl, and step c) further comprises the steps of:
(i) converting the compound of Formula (6a) into the compound of Formula (21a) 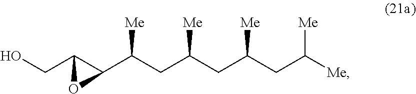
(ii) converting the compound of Formula (21a) into a compound of Formula (22a) 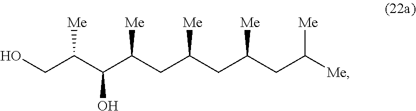
(iii) converting the compound of Formula (22a) into a compound of Formula (23a) 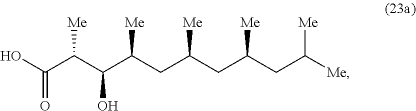
and
(iv) converting the compound of Formula (23a) into (+)-vittatalactone having the following structure:

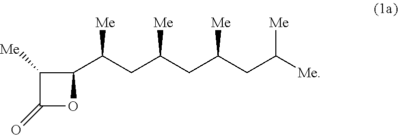

12. (canceled)

13. (canceled)

14. The method of claim 1, wherein R¹ is methyl.

15. The method of claim 1, wherein R¹ is methyl, and X² is a halogen; selected from among Cl, Br and I.

16. The method of claim 1, wherein the leaving group is an ester group, and/or the protecting group is a silyl ether.
